(19) United States
(12) Patent Application Publication
(10) Pub. No.: US 2026/0182863 A1
(43) Pub. Date: Jul. 2, 2026

(54) DEVICES, SYSTEMS, AND METHODS FOR MONITORING POSTURE VIA WEARABLE ARTICLES WITH FLEXIBLE CIRCUITS

(71) Applicant: Liquid Wire Inc., Portland, OR (US)

(72) Inventors: Michael Adventure Hopkins, Portland, OR (US); Austin Michael Clarke, Tigard, OR (US); Jorge E. Carbo, JR., Portland, OR (US)

(73) Assignee: Liquid Wire Inc., Portland, OR (US)

(21) Appl. No.: 19/418,286

(22) Filed: Dec. 12, 2025

Related U.S. Application Data
(63) Continuation of application No. PCT/US24/33555, filed on Jun. 12, 2024.
(60) Provisional application No. 63/507,484, filed on Jun. 12, 2023.

(51) Int. Cl. A61B 5/11; A61B 5/00

(52) U.S. Cl. CPC A61B 5/1116; A61B 5/6802; A61B 5/7271; A61B 5/7425

(57) ABSTRACT

A system configured to monitor a posture of a user is disclosed herein. The system can include a wearable article including a first flexible circuit including a trace including a deformable conductor, and a computing device configured to receive a first signal from the first flexible circuit, wherein the first signal is corresponds to a physical deformation of the first trace, determine a first electrical parameter based on the first signal, determine the posture of the user based on the determined electrical parameter, compare the determined posture of the user to a baseline for the user's posture, and cause a display communicably coupled to the computing device to present a visual representation of the comparison.

### CROSS-REFERENCE TO RELATED APPLICATIONS

The present application is a continuation of PCT Application No. PCT/US2024/033555, filed 12 Jun. 2024, which claims the benefit of priority from U.S. Provisional Patent Application No. 63/507,484, filed 12 Jun. 2023, the disclosures of which are hereby incorporated by reference in their entirety. All applications referenced herein are relevant to the subject matter disclosed herein and are hereby incorporated by reference in their entirety, regardless of the specific portion of the specification in which they are referenced.

### FIELD

The present disclosure is generally related to flexible circuits and, more particularly, is directed to flexible circuits that can be either integrated into wearable articles for the purposes of characterizing physical motions of a user of a wearable article.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, a system configured to monitor a posture of a user is disclosed. The system can include a wearable article including a first flexible circuit, wherein the first flexible circuit includes a first trace including a deformable conductor; and a computing device communicably coupled to the wearable article, wherein the computing device includes a processor and a memory configured to store instructions that, when executed by the processor, cause the processor to: receive a first signal from the first flexible circuit, wherein the first signal is corresponds to a physical deformation of the first trace; determine a first electrical parameter based on the first signal; determine the posture of the user based on the determined electrical parameter; compare the determined posture of the user to a baseline for the user's posture; and cause a display communicably coupled to the computing device to present a visual representation of the comparison.

In various aspects, a wearable article configured to monitor a posture of a user is disclosed. The wearable article can include a first flexible circuit, wherein the first flexible circuit includes a first trace including a deformable conductor, and wherein the wearable article is configured to be communicably coupled to a computing device, wherein the computing device includes a processor and a memory configured to store instructions that, when executed by the processor, cause the processor to receive a first signal from the first flexible circuit, wherein the first signal is corresponds to a physical deformation of the first trace, determine a first electrical parameter based on the first signal, determine the posture of the user based on the determined electrical parameter, compare the determined posture of the user to a baseline for the user's posture, and cause a display communicably coupled to the computing device to present a visual representation of the comparison.

In various aspects, a computer-implemented method of monitoring a posture of a user via a wearable article is disclosed. The wearable article can include a first flexible circuit, wherein the first flexible circuit includes a first trace including a deformable conductor. The method can include receiving, via a processor, a first signal from the first flexible circuit, wherein the first signal is corresponds to a physical deformation of the first trace, determining, via the processor, a first electrical parameter based on the first signal, determining, via the processor, a posture of the user based on the determined electrical parameter, comparing, via the processor, the determined posture of the user to a baseline for the user's posture, and causing, via the processor, a display communicably coupled to the processor to present a visual representation of the comparison.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a posture monitoring strain sensor system including a two-dimensional strain sensor, according to at least one non-limiting aspect of the present disclosure;

FIGS. 2A-E illustrate individual layers of a medium of the posture monitoring strain sensor system of FIG. 1, according to at least one non-limiting aspect of the present disclosure;

FIGS. 3A and 3B illustrate traces of a posture monitoring strain sensor system in a relaxed condition and a deformed condition, according to at least one non-limiting aspect of the present disclosure;

FIG. 4 illustrates another posture monitoring strain sensor, according to at least one non-limiting aspect of the present disclosure;

FIGS. 5A-D illustrate a posture monitoring flexible circuit configured to be integrated into a wearable article, according to at least one non-limiting aspect of the present disclosure;

FIGS. 5E and 5F illustrate an electrode configured for use with the flexible circuits and wearable articles disclosed herein is depicted, according to at least one non-limiting aspect of the present disclosure;

FIG. 6 illustrates a flow chart of a method of monitoring posture using a flexible circuit, in accordance with at least one non-limiting aspect of the present disclosure;

FIG. 7 illustrates a chart of an electrical parameter generated by a flexible circuit on an article worn by a user as their posture changes in time, according to at least one non-limiting aspect of the present disclosure;

FIG. 8 illustrates a wearable article featuring flexible circuits configured to monitor the posture of a user, according to at least one non-limiting aspect of the present disclosure.

FIG. 9 illustrates another wearable article featuring flexible circuits configured to monitor the posture of a user, according to at least one non-limiting aspect of the present disclosure.

FIGS. 10-12 illustrate several other wearable articles featuring flexible circuits configured to monitor the posture of a user, according to several non-limiting aspects of the present disclosure;

FIG. 13 illustrates another wearable article featuring flexible circuits configured to monitor the posture of a user, according to at least one non-limiting aspect of the present disclosure;

FIG. 14 illustrates a method of calibrating data generated by the flexible circuits of a wearable article, according to at least one non-limiting aspect of the present disclosure;

FIG. 15A illustrates a subject standing with a baseline posture.

FIG. 15B illustrates a subject standing with a Kyphosis-type posture relative to the baseline posture of FIG. 15A.

FIG. 15C illustrates a subject standing with a Lordosis-type posture relative to the baseline posture of FIG. 15A.

FIG. 15D illustrates a subject sitting with a baseline posture.

FIG. 15E illustrates a subject sitting with a Kyphosis-type posture relative to the baseline posture of FIG. 15A.

FIG. 15F illustrates a subject sitting with a Lordosis-type posture relative to the baseline posture of FIG. 15A.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. Furthermore, it is to be understood that such terms as “forward”, “rearward”, “left”, “right”, “upwardly”, “downwardly”, and the like are words of convenience and are not to be construed as limiting terms.

As used herein, the term “posture” shall refer to the position in which someone holds their body or a particular body part. For example, according to some non-limiting aspects described herein, posture can refer to the position in which a person's spine is maintained while sitting or standing. However, according to other non-limiting aspects, posture can refer to separate body parts, such as a person's head, shoulders, and/or hips. Moreover, according to other non-limiting aspects, the term posture can include a relative position of a person's body parts. For example, the term posture can refer to the relative position of a person's spine, head, shoulder, and/or hips. It shall be further appreciated that the term posture, as used herein, can refer to a dynamic posture—the position of one or more body parts during movement (e.g., walking, running, etc.)—and/or a static posture—the position of one or more body parts when are still (e.g., during sleep, while sitting, lying down, standing, etc.).

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

Although flexible and deformable electronic circuits have emerged as a means of innovating conventional electronics, such circuits are generally limited by flexion and fatigue. Thus, conventional circuits are not suitable for daily use applications where they will undergo repeated flexions. For example, conventional circuits would not be suitable in situations where a patient's motions should be monitored frequently, such as during a rehabilitation, and/or training. This includes the use of a flexible circuit to detect and classify a user's posture, which can enable correction and a reduction of the negative consequences of poor posture on the user's health. Moreover, as most conventional posture monitoring devices are rigid and configured to be adhered to the user's back, they are generally uncomfortable. Accordingly, there is a need for devices, systems, and methods for monitoring posture via wearable articles with flexible circuits. It shall be appreciated that, when made with the deformable conductors disclosed herein, a change in circuit geometry could lead to a subsequent change in electrical parameters generated across such flexible circuits. The varying electrical parameters generated by the flexible circuits disclosed herein could be used to characterize a structural parameter or condition of the circuit and thus, the posture of a user, as desired.

While certain electronic components typically have some inherent flexibility, that flexibility is typically constrained both in the amount the components can flex, their resilience in flexing, and the number of times the electronic components can flex before the electronic components deteriorate or break. Consequently, the utility of such electronic components in various environments may be limited, either by reliability or longevity or by the ability to function at all. Moreover, the lateral size of such components may result in additional stresses placed on the component.

The use of conductive gel, however, provides for electronic components that are flexible and deformable while maintaining resiliency. Moreover, in some embodiments the operational flexing, stretching, deforming, or other physical manipulation of a conductive trace formed from conductive gel may produce predictable, measurable changes in the electrical characteristics of the trace. By measuring the change in resistance or impedance of such a trace the change in length of the trace may be inferred. By combining the changes in lengths of multiple traces, the relative movement of points on a two-dimensional surface may be calculated.

A two-dimensional strain sensor has been developed that utilizes a network of conductive gel traces, the individual electrical characteristics of which translates to a relative length or other orientation of the trace. By combining the electrical characteristics, e.g., by triangulating or other mathematical process, the relative location of various points on a two-dimensional surface may be determined. By measuring such electrical characteristics repeatedly over time, the motion of the points may be determined, providing for the capacity for real-time motion capture of the points on the strain sensor. By scaling the network of traces and/or increasing the number of strain sensor and placing the strain sensors on an object, motion capture the object may be obtained in real-time.

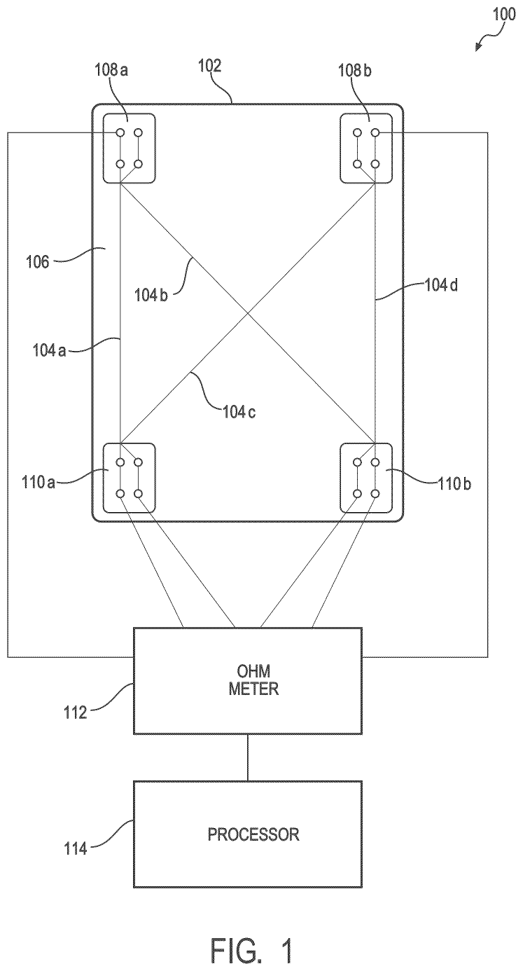

Referring now to FIG. 1, a view of a strain sensor system 100 including a two-dimensional strain sensor 102 is depicted in accordance with at least one non-limiting aspect of the present disclosure. As an example, the strain sensor system 100 can be configured similar to those disclosed in U.S. Provisional Patent Application No. 63/263,112, titled TWO DIMENSIONAL MOTION CAPTURE STRAIN GAUGE SENSOR, filed Oct. 10, 2021, the disclosures of which are hereby incorporated by reference in its entirety. The strain sensor 102 includes four traces 104a, 104b, 104c, 104d. Each trace 104a-104d is made of conductive gel, as disclosed in detail herein. The conductive gel is positioned on and encapsulated by a medium 106. Each trace 104a, 104b, 104c, 104d extends between and electrically couples one of two reference point 108a, 108b to an anchor point 110a, 110b. In the illustrated example, reference points 108a, 108b are not directly connected to one another and the anchor points anchor point 110a, 110b are not directly connected to one another.

The medium 106 specifically and the strain sensor 102 generally may be formed according to the techniques described herein or according to any other mechanism that exists or may be developed, including but not limited to injection molding, 3D printing, thermoforming, laser etching, die-cutting, and the like. The medium 106 may be formed of one of: a B-stage resin film, a C-stage resin film, an adhesive, a thermoset epoxy-based film, thermoplastic polyurethane (TPU), and/or silicone, among other suitable compounds or material. In an example, the medium 106 has tensile elongation of 550%; tensile modulus of 5.0 megapascals; recovery rate of 95%; thickness of 100 micrometers; a peel strength at 90 degrees of at least 1.0 kilonewtons per meter; a dielectric constant of 2.3 at 10 gigahertz; a dielectric dissipation factor of 0.0030 at 10 gigahertz; a breakdown voltage of 7.0 kilovolts at a thickness of 80 micrometers; a heat resistance that produces no change in an environment of 260 degrees Celsius for 10 cycles in a nitrogen atmosphere; and chemical resistance producing no change to the medium 106 after 24 hours immersion in any of NaOH, Na2CO3, or copper etchant.

Details of an example medium 106 are disclosed in U.S. Patent Application Publication No. 2020/0381349, “CONTINUOUS INTERCONNECTS BETWEEN HETEROGENEOUS MATERIALS”, Ronay et al., which is incorporated by reference herein in its entirety.

The strain sensor 102 is configured to identify changes in the relative positions of the reference points 108a, 108b based on a change in impedance/resistance of one or more of the traces 104a, 104b, 104c, 104d. In particular, the strain sensor 102 is configured to determine the relative position according to the Cartesian system (x,y) on a plane defined by the medium 106 of a given reference point 108a, 108b in relation to the two anchor points 110a, 110b to which the reference point 108a, 108b is coupled via an associated trace 104a, 104b, 104c, 104d. Thus, for instance, the relative position of the reference point 108a may be determined by one or, inferentially, both of: determining the length at any given time of the trace 104a and the trace 104b and/or by determining the relative position (x,y) of the anchor points 110a, 110b.

The length of the traces 104a, 104b may be determined as a function of resistance and/or impedance of the given trace 104a, 104b, 104c, 104d as measured between the reference point 108a, 108b and the anchor point 110a, 110b that is coupled by the trace 104a, 104b, 104c, 104d. In the illustrated example, the strain sensor system 100 includes an electronic parameter sensor 112 operatively coupled to a processor 114. The electronic parameter sensor 112 may be any device that is configured to detect or otherwise measure an electronic property, such as resistance, capacitance, inductance, etc. As such, in various examples, the electronic parameter sensor 112 may be an ohm meter or a resistance signal reader. Further, the electronic parameter sensor 112 and the processor 114 may be separate components or integrated together. In such an example, the processor 114 may be part of a chipset or package that incorporates resistance signal reading and recording capabilities. In still yet other examples, an analog to digital signal processor may be utilized to convert an analog resistance signal to a digital signal, which may be received by the processor 114. In examples where a remote processor is configured to receive signals from the strain sensor 102, a wireless communication component integrated to the sensor may be configured to provide signals to the processor 114.

While the strain sensor system 100 as illustrated includes the electronic parameter sensor 112 and the processor 114, it is to be recognized and understood that one or both of the electronic parameter sensor 112 and the processor 114 may be remote to the rest of the strain sensor system 100 and/or cloud computing assets, etc. Moreover, in various examples the electronic parameter sensor 112 and/or the processor 114 may be integrated into the strain sensor 102 itself or may be components to which the strain sensor 102 is operatively coupled, as illustrated. In examples where the processor 114 and/or the electronic parameter sensor 112 are remote to the strain sensor 102, a wireless communication module may be incorporated into the strain sensor 102 to provide data to the electronic parameter sensor 112 and/or processor 114.

In various examples, the processor 114 does not require a calibrated or predetermined relationship of impedance of a given trace 104a, 104b, 104c, 104d to determine the relative position of a reference point 108a, 108b and/or a relative position of an anchor point 110a, 110b. In such an example, the processor 114 may determine the relative location (x,y) on the medium 106 of the reference point 108a by determining location of the reference point 108a relative to the determined location (x,y) of each of the anchor points 110a, 110b to which the traces 104a, 104b are coupled. In such an example, the location variables x and y of the reference point 108a may be determined by the processor 114 according to the following equations:

$$\begin{matrix}Equation 1 : X - Coordinate of the Reference Point & \\ x = \frac{ℓ}{\partial } ( {x}_{a} - {x}_{b} ) \pm \frac{h}{\partial } ( {y}_{a} - {y}_{b} ) + {x}_{b} & \end{matrix}$$

$$\begin{matrix}Equation 2 : Y - Coordinate of the Reference Point & \\ y = \frac{ℓ}{\partial } ( {y}_{a} - {y}_{b} ) ∓ \frac{h}{\partial } ( {x}_{a} - {x}_{b} ) + {y}_{b} & \end{matrix}$$

$$h = \sqrt{{r}_{b}^{2} - {ℓ}^{2}} \qquad Equation 3$$

$$ℓ = \frac{{r}_{b}^{2} - {r}_{a}^{2} + {\partial }^{2}}{2 \partial } \qquad Equation 4$$

$$\partial = \sqrt{{( {x}_{a} - {x}_{b} )}^{2} + {( {y}_{a} - {y}_{b} )}^{2}} \qquad Equation 5$$

In the above equations, r is the impedance for a given trace 104a, 104b as measured by the electronic parameter sensor 112 and provided to the processor 114. By applying the same equations in the same manner for the reference point 108b, but for the traces 104c, 104d, the position of each of the reference points 108a, 108b may be determined. By performing the calculations a relatively high frequency, e.g., at least once per second, or at least fifteen (15) times per second, or at least twenty-four times per second, etc., the strain sensor system 100 may obtain a real-time determination of the relative positions of the reference points 108a, 108b and, therefore, the amount and rate of movement of the reference points 108a, 108b.

While the strain sensor system 100 is described with respect the measurement of resistance or impedance, it is to be recognized and understood that any electrical measurement may be applied on a similar basis. Thus, for instance, the traces 104a, 104b, 104c, 104d may have or may be configured to have an inductance, a capacitance, or other measureable electronic property that may be changed based on a deformation of the trace. Consequently, while an electronic parameter sensor 112 is described and illustrated, it is to be recognized and understood that any electronic meter configured to sense and measure the relevant electronic property may be utilized in addition to or instead of the electronic parameter sensor 112 in a manner consistent with this disclosure.

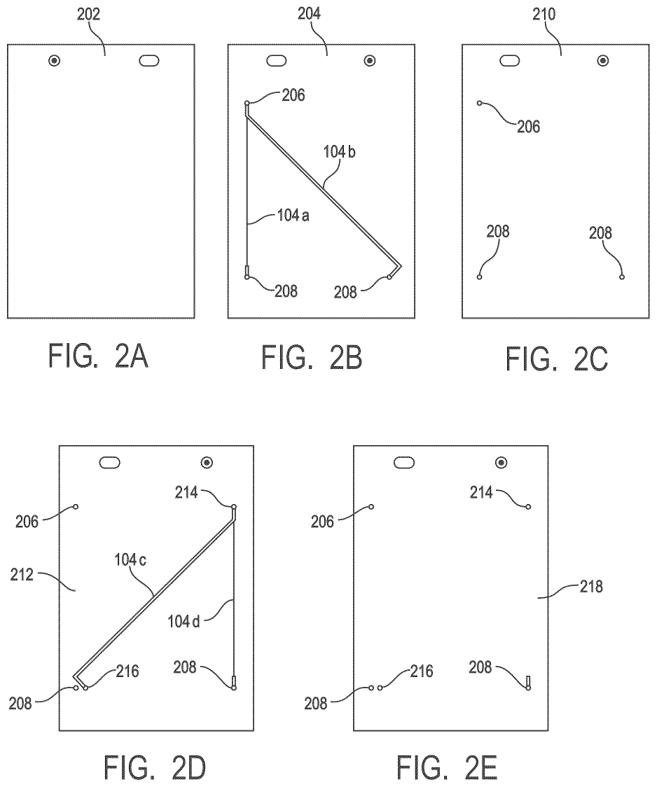

As will be described in further detail with reference to FIGS. 2A-E, the strain sensor 102 can include a multi-layered construction, consisting of a substrate layer 202 (FIG. 2A), at least one patterned layer 204, 212 (FIGS. 2B and 2D), and at least one encapsulation layer 210, 218 (FIGS. 2C and 2E). For example, according to the non-limiting aspect of FIG. 1, two of the traces 104a, 104b can be deposited on a first patterned layer 204 (FIG. 2B) of the strain sensor 102, and two of the traces 104c, 104d can be deposited on a second patterned layer 212 (FIG. 2D) of the strain sensor 102. Two or more of the traces 104a-d, for example, can be electrically coupled through various layers of the strain sensor 102 by vias. One or more of the layers 202, 204, 210, 212, 218 (FIGS. 2A-E) can include a stretchable epoxy-based material, such as a Lubrizol Estane 58000 series (e.g., 58238), amongst others. Other examples of materials with adhesive properties include some thermally activated adhesives like polyurethane (PU) adhesives (e.g., from Bemis or Framis), thermoset adhesives with different chemistry such as some silicones, acrylics or others, and any pressure sensitive adhesive of any chemistry, etc.

It shall be appreciated that such stretchable epoxy-based materials may provide a self-adhesive surface conducive to bonding electronic components to each layer 202, 204, 210, 212, 218 (FIGS. 2A-E), and for bonding the various layers 202, 204, 210, 212, 218 (FIGS. 2A-E) of the strain sensor 102 to one another. Such stretchable epoxy-based materials may be transparent, which explains why the traces 104a-d of the strain sensor 102 of FIG. 1 can be seen through the encapsulation layers 210, 218 (FIG. 2A). However, according to other non-limiting aspects, one or more layers 202, 204, 210, 212, 218 (FIGS. 2A-E) may be opaque or semi-transparent.

According to other non-limiting aspects, the strain sensor 102 can employ a two-layer construction techniques, similar to those disclosed in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, filed Feb. 25, 2022, the disclosure of which is herein incorporated by reference in its entirety. For example, at least one of the patterned layers 204, 212 (FIGS. 2B and 2D) can be omitted from the assembly and the deformable conductor that defines the traces 104a-d can be deposited directly onto a substrate layer 202 (FIG. 2A) and/or the encapsulation layer, such as encapsulation layer 210 (FIG. 2C). Of course, according to other non-limiting aspects, the all of the traces 104a-d can be deposited on a single patterned layer 204 (FIG. 2B) or the substrate layer 202 (FIG. 2A), itself.

FIGS. 2A-2E are depictions of individual layers of the medium 106 of the strain sensor 102, in an example embodiment. In the example of FIGS. 2A-2E, the strain sensor 102 is a laminate structure in that individual layers of the medium 106 are separately formed, stacked, and unitized together to create the medium 106 as a whole. The layers may be formed according to iterative stencil-in-place processes described in U.S. Patent Application Publication No. 2020/0066628, titled “STRUCTURES WITH DEFORMABLE CONDUCTORS,” filed Aug. 22, 2019, the disclosure of which is hereby incorporated by reference in its entirety. However, as noted above, the formation of the strain sensor 102 as a laminate structure is merely an example of a strain sensor 102 construction and not limitation. Accordingly, it shall be appreciated that any suitable technique for making the strain sensor 102 may be applied instead of or in addition to the process of making the strain sensor 102 as a laminate structure. The depictions of the layers are looking along a major axis of the strain sensor 102 and are thus either a top or bottom view of the layer relative to the perspective of FIG. 1.

FIG. 2A is substrate layer 202. The substrate layer 202 is formed of the material of the medium 106 and eventually has traces 104a, 104b placed thereon but is otherwise featureless and may, in various examples, provide insulation for and/or containment of the conductive gel.

FIG. 2B is a first patterned layer 204. The first patterned layer 204 is formed of the material of the medium 106 and includes the traces 104a, 104b, e.g., formed as channels that contain conductive gel formed in the medium 106. Additionally, a first reference via 206 and first anchor vias 208 are operatively coupled to the respective traces 104a, 104b and provide electrical access to the traces 104a, 104b through various layers of the strain sensor 102. The vias 206, 208 may be formed from conductive gel or any suitable conductor.

FIG. 2C is an insulation layer 210. The insulation layer 210 is formed of the material of the medium 106 and includes the first reference via 206 and the first anchor vias 208, which extend through the insulation layer 210.

FIG. 2D is a second patterned layer 212. The second patterned layer 212 is formed of the material of the medium 106 and includes the traces 104c, 104d, e.g., formed as channels that contain conductive gel formed in the medium 106. The first reference via 206 and the first anchor vias 208 extend through the second patterned layer 212, and a second reference via 214 and second anchor vias second anchor via 216 are operatively coupled to traces 104c, 104d.

FIG. 2E is an encapsulation layer 218. The encapsulation layer 218 is formed of the material of the medium 106 and includes the first reference via 206, the first anchor vias 208, the second reference via 214, and the second anchor vias 216, all of which are exposed beyond the medium 106 to enable the strain sensor 102 to be operatively coupled to the electronic parameter sensor 112, as shown in FIG. 1.

The various layers are presented for illustration and not limitation and it is to be recognized and understood that any of a variety of additional or alternative layers may be incorporated into the laminate structure as desired. The laminate structure may incorporate at least one substrate layer onto which conductive gel is positioned, at least one patterned layer that forms at least one trace, and at least one encapsulation layer that seals the trace or other component of the laminate structure. The laminate structure may further include: a stencil layer, e.g., for when a stencil-in-place manufacturing process is utilized; a conductive layer for, e.g., a relatively high-powered bus, sensor, ground plane, shielding, etc.; an insulation layer, e.g., between a substrate layer, a conductive layer, a stencil layer, and/or an encapsulation layer, that primarily insulates traces or conductive layers from one another; an electronic component not necessarily formed according to the processes disclosed herein, e.g., a surface mount capacitor, resistor, processor, etc.; vias for connectivity between layers; and contact pads.

The collection of layers of the laminate structure may be referred to as a “stack”. A final or intermediate structure may include at least one stack (or multiple stacks, e.g., using modular construction techniques) that has been unitized. Additionally or alternatively, the structure could comprise one or more unitized stacks with at least one electronic component. A laminate assembly may comprise multiple laminate structures, e.g., in a modular construction. The assembly may utilize island architecture including a first laminate structure (the “island”), which may typically but not exclusively be itself a laminate structure populated with electric components, or a laminate structure that is, e.g., a discrete sensor, with the first laminate structure adhered to a second laminate structure including, e.g., traces and vias configured like a traditional printed circuit board (“PCB”), e.g., acting as the pathways for signals, currents or potentials to travel between the island(s) and other auxiliary structures, e.g., sensors.

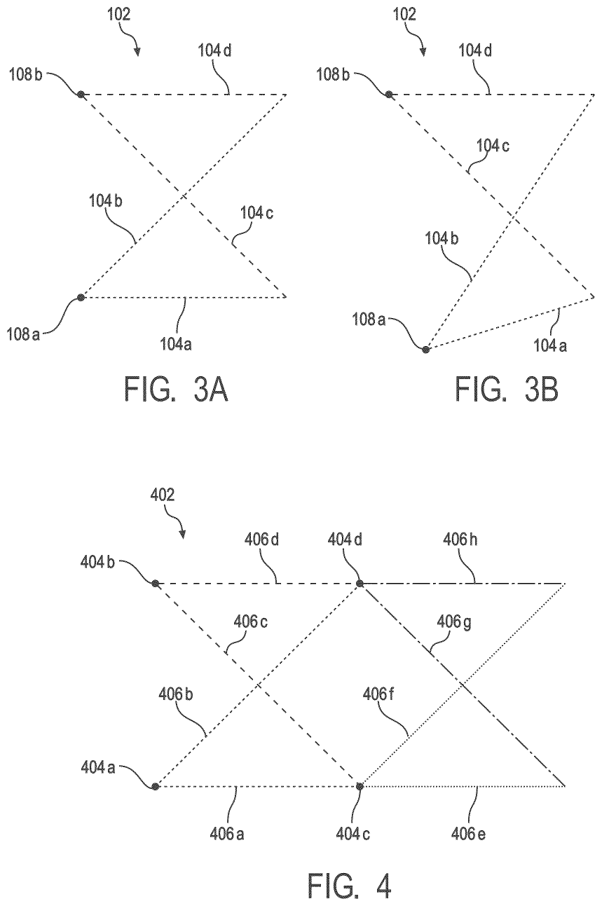

FIGS. 3A and 3B are abstract depictions of the traces of the strain sensor 102 in a relaxed and deformed configuration, respectively. The strain sensor 102 is considered to be in the relaxed configuration when an outside force is not acting on the strain sensor 102 such that the strain sensor 102 deforms through stretching, flexing, etc. The strain sensor 102 is considered to be in the deformed configuration when an outside for is acting on the strain sensor 102 such that the strain sensor 102 deforms through stretching, flexing, etc., and, as a result, one or more of the traces 104a, 104b, 104c, 104d lengthen or contract relative to their length in the relaxed configuration. It is noted that FIGS. 3A and 3B are described in a two-dimensional plane, but it is to be recognized and understood that the principles described with respect to two dimensions apply as well to three dimensional strain placed on the strain sensor 102.

In the illustrated example, in the relaxed configuration the traces 104a, 104d are of substantially equal length, e.g., within five (5) percent, and, as a result, of approximately equal resistance or impedance. Similarly, the traces 104b, 104c are similarly of substantially equal length and, as a result, of approximately equal distance. In such a circumstance, the processor 114 would determine that the relative (x, y) location of the reference points 108a, 108b are in their relaxed state.

In the deformed configuration, an outside force causes the reference point 108a to move relative to the reference point 108b. In the illustrated example, the length, and consequently, resistance of the traces 104c, 104d have not substantially changed, resulting in the processor 114 being configured to determine that, at least on a relative basis, strain has not been placed on the strain sensor 102 proximate the reference point 108b. However, the length, and consequently, the resistance of the traces 104a, 104b have changed, in the case of trace 104a to shorten and in the case of trace 104b to lengthen relative to the length of those traces 104a, 104b in the relaxed state. Consequently, the processor 114 would be configured to determine that a strain has been placed on the strain sensor 102 proximate the reference point 108a.

Strain placed on the strain sensor 102 at different locations would result in different deformation of the strain sensor 102 and, consequently, different lengthening or shortening of the traces 104a, 104b, 104c, 104d than illustrated here. Moreover, while the length of two traces is shown as being constant, any or all of the traces 104a, 104b, 104c, 104d may change length and, consequently, measured resistance. Moreover, the strain sensor 102 may be sensitive to multiple forces placed on the strain sensor 102 to the extent that those different forces manifest at different locations on the strain sensor 102.

FIG. 4 is an abstract depiction of a strain sensor 402, in an example embodiment. In contrast to the strain sensor 102, the strain sensor 402 includes four reference points 404a, 404b, 404c, 404d. In such an example, the reference points 404c, 404d may function as de facto anchor points in relation to the reference points 404a, 404b. Consequently, the resistance over the trace 406a may be measured from reference point 404a to reference point 404c, and so forth.

The relative position of each reference point 404a, 404b, 404c, 404d are each determined by two of the traces 406. For the sake of clarity, the traces 406 associated with each reference point 404a, 404b, 404c, 404d are denoted by a particular dashed line. Thus, the relative position (x,y) of the reference point 404a is determined based on the resistance of the traces 406a, 406b, the relative position of the reference point 404c is based on the resistance of the traces 406e, 406f, and so forth. The principles disclosed herein are readily expandable to any number of reference points over any given area. The number of inputs on the electronic parameter sensor 112 or ohm meters may be expanded proportionally along with the processing resources of the processor 114.

Moreover, it is to be recognized and understood that number of traces associated with a given reference point may expand based on the available traces. In various examples, the relative position of a reference point may be determined based on three or more traces rather than only two, with the equations described above expanded to incorporate the additional traces. However, in further examples the additional traces beyond two for each reference point 404 may be treated as redundant traces. Thus, the processor 114 may only utilize two traces to determine the relative position of a given reference point, but if a trace to a reference point 404 breaks then the processor 114 may utilize a different, unbroken trace to determine the relative position of the reference point 404.

The inclusion of multiple reference points 404 in a strain sensor and/or multiple strain sensor may provide for the creation of a real-time three dimensional model of a larger object. Thus, for instance, a wearable article may have traces extending throughout the wearable article, with the traces coupled to many reference points distributed throughout the wearable article. By regularly determining the relative position of each reference point, the processor 114 may readily create a three-dimensional model of the wearable article based on the change in relative position of each reference point to neighboring reference points.

Adaptation of the strain sensors disclosed herein to various use cases may result in the length of traces being optimized for the conditions of the wearable article or other article to which the strain sensor is attached. Thus, for instance, some traces may be relatively longer and the reference points spaced apart in certain locations that would not be expected to have strain placed thereon, while other traces may be relatively shorter and reference points spaced closer together in locations that may be expected to have strain placed thereon, e.g., at an elbow of a sleeve.

The electrically conductive compositions, such as conductive gels, comprised in the articles described herein can, for example, have a paste like or gel consistency that can be created by taking advantage of, among other things, the structure that gallium oxide can impart on the compositions when gallium oxide is mixed into a eutectic gallium alloy. When mixed into a eutectic gallium alloy, gallium oxide can form micro or nanostructures that are further described herein, which structures are capable of altering the bulk material properties of the eutectic gallium alloy.

As used herein, the term “eutectic” generally refers to a mixture of two or more phases of a composition that has the lowest melting point, and where the phases simultaneously crystallize from molten solution at this temperature. The ratio of phases to obtain a eutectic is identified by the eutectic point on a phase diagram. One of the features of eutectic alloys is their sharp melting point.

According to some non-limiting aspects, the strain sensor 102 of FIGS. 2A-2E can be formed using any of the methods described in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety. For example, according to some non-limiting aspects, the stencil layer can be omitted, as traces made from deformable conductors can be deposited directly on a substrate layer 202, as described in reference to FIG. 2A, and subsequently encapsulated without including a stencil layer in the final layup assembly. For example, the properties of the deformable conductive material and/or the properties of the layers surrounding the patterns of the deformable conductive material may be adjusted and/or optimized to ensure that the patterns of deformable conductive material heal upon unitization of the surrounding layers. For example, the deformable conductive material may be optimized to have a viscosity such that the deformable conductive material is able to heal upon unitization of the layers but not such that the deformable conductive material overly deforms and does not achieve the intended pattern. As another example, adhesive characteristics and/or viscosity of the deformable conductive material may be optimized such that it remains on the substrate layer upon removal of the removable stencil 50 and but does not adhere to the channels 504, 506 of the stencil thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pas and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

The electrically conductive compositions described herein can have any suitable conductivity, such as a conductivity of from about 2×10⁵ S/m to about 8×10⁵ S/m.

The electrically conductive compositions described herein can have ay suitable melting point, such as a melting point of from about −20° C. to about 10° C., about −10° C. to about 5° C., about −5° C. to about 5° C. or about −5° C. to about 0° C.

The electrically conductive compositions can comprise a mixture of a eutectic gallium alloy and gallium oxide, wherein the mixture of eutectic gallium alloy and gallium oxide has a weight percentage (wt %) of between about 59.9% and about 99.9% eutectic gallium alloy, such as between about 67% and about 90%, and a wt % of between about 0.1% and about 2.0% gallium oxide such as between about 0.2 and about 1%. For example, the electrically conductive compositions can have about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy, and about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide.

The eutectic gallium alloy can include gallium-indium or gallium-indium-tin in any ratio of elements. For example, a eutectic gallium alloy includes gallium and indium. The electrically conductive compositions can have any suitable percentage of gallium by weight in the gallium-indium alloy that is between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

The electrically conductive compositions can have a percentage of indium by weight in the gallium-indium alloy that is between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

The eutectic gallium alloy can include gallium and tin. For example, the electrically conductive compositions can have a percentage of tin by weight in the alloy that is between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The electrically conductive compositions can comprise one or more micro-particles or sub-micron scale particles blended with the eutectic gallium alloy and gallium oxide. The particles can be suspended, either coated in eutectic gallium alloy or gallium and encapsulated in gallium oxide or not coated in the previous manner, within eutectic gallium alloy. The micro- or sub-micron scale particles can range in size from nanometer to micrometer and can be suspended in gallium, gallium-indium alloy, or gallium-indium-tin alloy. Particle to alloy ratio can vary and can change the flow properties of the electrically conductive compositions. The micro and nanostructures can be blended within the electrically conductive compositions through sonication or other suitable means. The electrically conductive compositions can include a colloidal suspension of micro and nanostructures within the eutectic gallium alloy/gallium oxide mixture.

The electrically conductive compositions can further include one or more micro-particles or sub-micron scale particles dispersed within the compositions. This can be achieved in any suitable way, including by suspending particles, either coated in eutectic gallium alloy or gallium and encapsulated in gallium oxide or not coated in the previous manner, within the electrically conductive compositions or, specifically, within the eutectic gallium alloy fluid. These particles can range in size from nanometer to micrometer and can be suspended in gallium, gallium-indium alloy, or gallium-indium-tin alloy. Particle to alloy ratio can vary, in order to, among other things, change fluid properties of at least one of the alloys and the electrically conductive compositions. In addition, the addition of any ancillary material to colloidal suspension or eutectic gallium alloy in order to, among other things, enhance or modify its physical, electrical or thermal properties. The distribution of micro and nanostructures within the at least one of the eutectic gallium alloy and the electrically conductive compositions can be achieved through any suitable means, including sonication or other mechanical means without the addition of particles. In certain embodiments, the one or more micro-particles or sub-micron particles are blended with the at least one of the eutectic gallium alloy and the electrically conductive compositions with wt % of between about 0.001% and about 40.0% of micro-particles, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40.

The one or more micro- or sub-micron particles can be made of any suitable material including soda glass, silica, borosilicate glass, quartz, oxidized copper, silver coated copper, non-oxidized copper, tungsten, super saturated tin granules, glass, graphite, silver coated copper, such as silver coated copper spheres, and silver coated copper flakes, copper flakes, or copper spheres, or a combination thereof, or any other material that can be wetted by the at least one of the eutectic gallium alloy and the electrically conductive compositions. The one or more micro-particles or sub-micron scale particles can have any suitable shape, including the shape of spheroids, rods, tubes, a flakes, plates, cubes, prismatic, pyramidal, cages, and dendrimers. The one or more micro-particles or sub-micron scale particles can have any suitable size, including a size range of about 0.5 microns to about 60 microns, as about 0.5 microns, about 0.6 microns, about 0.7 microns, about 0.8 microns, about 0.9 microns, about 1 microns, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

The electrically conductive compositions described herein can be made by any suitable method, including a method comprising blending surface oxides formed on a surface of a eutectic gallium alloy into the bulk of the eutectic gallium alloy by shear mixing of the surface oxide/alloy interface. Shear mixing of such compositions can induce a cross linked microstructure in the surface oxides; thereby forming a conducting shear thinning gel composition. A colloidal suspension of micro-structures can be formed within the eutectic gallium alloy/gallium oxide mixture, for example as, gallium oxide particles and/or sheets.

The surface oxides can be blended in any suitable ratio, such as at a ratio of between about 59.9% (by weight) and about 99.9% eutectic gallium alloy, to about 0.1% (by weight) and about 2.0% gallium oxide. For example percentage by weight of gallium alloy blended with gallium oxide is about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy while the weight percentage of gallium oxide is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide. In embodiments, the eutectic gallium alloy can include gallium-indium or gallium-indium-tin in any ratio of the recited elements. For example, a eutectic gallium alloy can include gallium and indium.

The weight percentage of gallium in the gallium-indium alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

A eutectic gallium alloy can include gallium, indium, and tin. The weight percentage of tin in the gallium-indium-tin alloy can be between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.4%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The weight percentage of gallium in the gallium-indium-tin alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium-tin alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

One or more micro-particles or sub-micron scale particles can be blended with the eutectic gallium alloy and gallium oxide. For example, the one or more micro-particles or sub-micron particles can be blended with the mixture with wt % of between about 0.001% and about 40.0% of micro-particles in the composition, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40. In embodiments the particles can be soda glass, silica, borosilicate glass, quartz, oxidized copper, silver coated copper, non-oxidized copper, tungsten, super saturated tin granules, glass, graphite, silver coated copper, such as silver coated copper spheres, and silver coated copper flakes, copper flakes or copper spheres or a combination thereof, or any other material that can be wetted by gallium. In some embodiments the one or more micro-particles or sub-micron scale particles are in the shape of spheroids, rods, tubes, a flakes, plates, cubes, prismatic, pyramidal, cages, and dendrimers. In certain embodiments, the one or more micro-particles or sub-micron scale particles are in the size range of about 0.5 microns to about 60 microns, as about 0.5 microns, about 0.6 microns, about 0.7 microns, about 0.8 microns, about 0.9 microns, about 1 microns, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

It shall be appreciated that, due to the aforementioned composition, a deformable conductor shall not only be flexible but also capable of being stretched. Although many materials may be capable of some degree of flexion, the deformable conductors described herein have the aforementioned characteristics. Such characteristics enable the composition of the deformable conductors to be rearranged as the conductor stretches to maintain conductivity, which promotes stretchability while preserving electrical conductivity. In other words, the length of the deformable conductor can be significantly extended and the width of the deformable conductor significantly reduced without breaking electrical conductivity. Thus, beyond limited flexions, the deformable conductors can remain electrically functional when stretched, bent, and/or twisted.

Moreover, because the aforementioned compositions can include micro-particles and/or sub-micron scale particles suspended within an electrically conductive medium, the deformable conductors disclosed herein can be easily wetted to a substrate layer and/or encapsulation layer of the layup. It shall be appreciated that “wettability” can include the ability of the deformable conductor to spread over a surface, in accordance with the contact angle between the deformable conductor and the surface. Surface energy will decrease proportionally relative to the contact angle. It shall be further appreciated that conventional liquid metals, such as gallium alloys, can be difficult to wet and thus, difficult to pattern on substrates and other surfaces. Whereas particulates have been conventionally perceived as impurities, along with the aforementioned viscosities, the deformable conductors disclosed herein can implement the particles within the conductive medium to enhance wettability without compromising conductivity. Accordingly, unlike conventional liquid metals, the deformable conductors disclosed herein can be easily deposited on a surface in intricate patterns. As previously discussed, the particles can be suspended, either coated in eutectic gallium alloy or gallium and encapsulated in gallium oxide or within the eutectic gallium alloy.

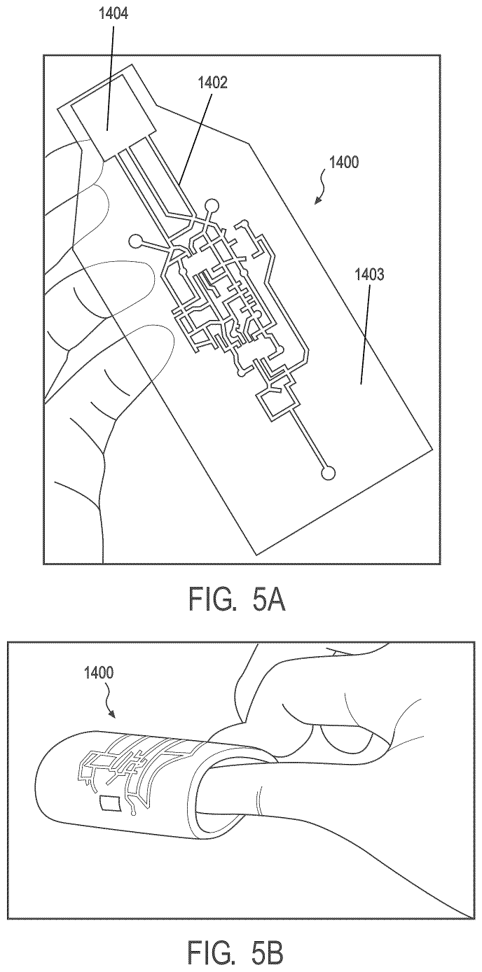

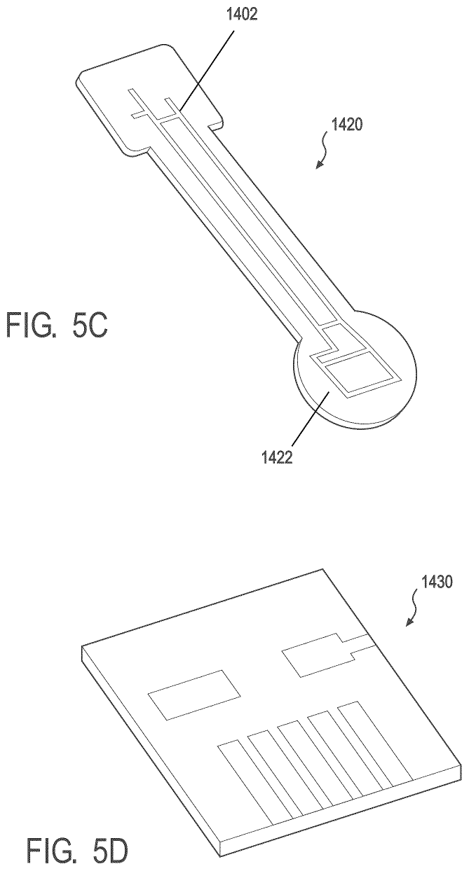

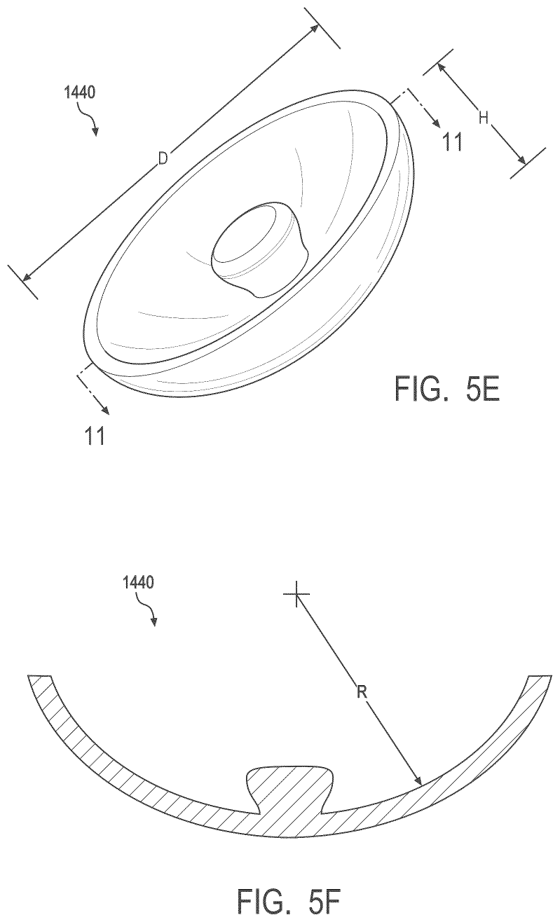

Referring now to FIGS. 5A and 5B, a flexible circuit 1400 configured for use with the articles disclosed herein is depicted according to at least one non-limiting aspect of the present disclosure. For example, according to the non-limiting aspect, a flexible circuit 1400 that comprises traces made from deformable conductors is depicted in a relaxed condition. However, according to the non-limiting aspect of FIG. 5B, the flexible circuit 1400 has been significantly deformed and is in a stressed condition. Accordingly, electrical parameters generated by the traces of deformable conductors will vary and the traces lengthen, due to the aforementioned nature of the deformable conductor. Thus, the flexible circuit 1400 of FIGS. 5A and 5B synthesizes the concepts described herein and is suitable for implementation via the wearable articles, systems, and methods for characterizing physical motions of a user, as described herein.

Referring now to FIGS. 5C and 5D, several electronic components 1420, 1430 configured for use with the flexible circuits disclosed herein are depicted in accordance with at least one aspect of the present disclosure. For example, the electronic components 1420, 1430 can be used in conjunction with strain sensor systems similar to the strain sensor system 100 of FIG. 1. The electronic components 1420, 1430 can be either attached to a laminate structure that encapsulates deformable conductors and/or, as in the case of the electronic component 1420 of FIG. 5C, can be constructed from deformable conductors themselves. According to some non-limiting aspects, for example, the electronic component 1420 of FIG. 5C can include one or more traces 1402 formed from a deformable conductor deposited on a medium 1403 and can be constructed in accordance with the techniques disclosed in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, and/or International Patent Application No. PCT/US2019/047731 titled STRUCTURES WITH DEFORMABLE CONDUCTORS, filed Aug. 22, 2019, the disclosures of which are hereby incorporated by reference in their entireties.

For example, as previously described in reference to FIGS. 1-4, the deformable conductor 1402 can include a conductive gel that is integrated into a multi-layered medium 1403, such as a laminate structure, enclosed with at least one encapsulation layer that seals the trace 1402 or other component of the laminate structure 1403. The laminate structure 1403 may further include: at least one stencil layer, e.g., for when a stencil-in-place manufacturing process is utilized; a conductive layer for, e.g., a relatively high-powered bus, sensor, ground plane, shielding, etc.; an insulation layer, e.g., between a substrate layer, a conductive layer, a stencil layer, and/or an encapsulation layer, that primarily insulates traces or conductive layers from one another; an electronic component not necessarily formed according to the processes disclosed herein, e.g., a surface mount capacitor, resistor, processor, etc.; vias for connectivity between layers; and contact pads.

The collection of layers of the laminate structure 1403 may be referred to as a “stack”. A final or intermediate structure may include at least one stack (or multiple stacks, e.g., using modular construction techniques) that has been unitized. Additionally or alternatively, the structure 1403 could comprise one or more unitized stacks with at least one electronic component. A laminate assembly 1403 may comprise multiple laminate structures, e.g., in a modular construction. The assembly may utilize island architecture including a first laminate structure (the “island”), which may typically but not exclusively be itself a laminate structure populated with electric components, or a laminate structure that is, e.g., a discrete sensor, with the first laminate structure adhered to a second laminate structure including, e.g., traces and vias configured like a traditional printed circuit board (“PCB”), e.g., acting as the pathways for signals, currents or potentials to travel between the island(s) and other auxiliary structures, e.g., sensors.

Additionally, the flexible circuit 1400 of FIGS. 5A-D can further include one or more sensors (e.g., sensors 102, 402 of FIGS. 1-4) and/or other electronic components (e.g., IMU's, processors, force sensors, inductive coil sensors, temperature sensors, etc.). The electronic components can be electrically coupled using flexible circuits composed of the deformable conductors 1402, as previously disclosed. According to some non-limiting aspects, at least one portion of the deformable conductors 1402 can be configured as a bus portion of the flexible circuit 1400 and/or a strain sensor portion of the flexible circuit 1400.

In reference of FIG. 5C, according to other non-limiting aspects, one or more portions 1422 of a flexible circuit 1420 can be configured as a pressure sensor, including any of those described in International Patent Application No. PCT/US2021/071374, titled WEARABLE ARTICLE WITH FLEXIBLE INDUCTIVE PRESSURE SENSOR, filed Sep. 3, 2021, U.S. Provisional Application No. 63/270,589, titled FLEXIBLE THREE-DIMENSIONAL ELECTRONIC COMPONENT, filed Oct. 22, 2021, and U.S. Provisional Application No. 63/272,487, titled DEVICES, SYSTEMS, AND METHODS FOR MAKING AND USING A FLUID-FILLABLE CIRCUIT, filed Oct. 27, 2021, the disclosures of which are hereby incorporated by reference in its entirety. For example, according to the non-limiting aspect of FIG. 5C, the one or more portions 1422 of the flexible circuit 1420 can be configured as a coil that can be biased relative to a conductive plane integrated within a wearable article (e.g., mounting the conductive plane on foam or within a bladder filled with compressible fluid, etc.). As a distance between the conductive plane and the coil of the one or more portions 1422 of the flexible circuit 1420 changes, a difference in an electrical parameter (e.g., electromagnetic inductance) can be detected, for example, via a capacitor of a resistor, inductor, capacitor (“RLC”) circuit, as disclosed in International Patent Application No. PCT/US2021/071374, titled WEARABLE ARTICLE WITH FLEXIBLE INDUCTIVE PRESSURE SENSOR, filed Sep. 3, 2021, U.S. Provisional Application No. 63/270,589. Accordingly, as the inductive coil of the one or more portions 1422 of the flexible circuit 1420 is depressed and/or extended, an electrical parameter (e.g., an electromagnetic inductance, etc.) generated by that portion 1422 of the flexible circuit 1420 will vary and corresponding signals can be transmitted via the circuits to the processor 114 (FIG. 1) for characterization of swelling at the location at which the portion 1422 is positioned. As such, the one or more portion 1422 of the flexible circuit 1420 configured as an inductive pressure sensor can be configured to monitor swelling in a specific portion of the joint and/or appendage, as previously disclosed.

According to the non-limiting aspect of FIG. 5D, a flexible circuit 1430 can be configured for “spot” monitoring in a particular location of the wearable article. For example, the flexible circuit 1430 of FIG. 5D can be configured to function as a temperature sensor and/or a pressure sensor to monitor, for example, blood flow and/or swelling, as previously disclosed.

It shall be appreciated that, due to the flexible nature of the deformable conductors 1402 and medium 1403, the flexible circuits 1400, 1420, 1430 can be imbued with a tremendous amount of flexibility relative to conventional circuits. For example, according to the non-limiting aspect of FIG. 5A, a flexible circuit 1400 is at rest and unstrained. As such, when a current is introduced through the traces formed by the deformable conductors 1402, the flexible circuit will generate a plurality of electrical parameters at rest (e.g., an inductance, a resistance, a voltage drop, a capacitance, and/or an electromagnetic field, etc.). However, according to the non-limiting aspect of FIG. 5B, the flexible circuit 1400 can essentially be folded in half—and, according to other non-limiting aspects, coiled and/or twisted—without introducing discontinuities between traces and/or electronic components. Of course, as the flexible circuit 1400 undergoes such deformations, it will the plurality of electrical parameters generated by the flexible circuit 1400 under varying degrees of stress will differ from those the flexible circuit 1400 generates at rest. According to some non-limiting aspects, the flexible circuit 1400, including the fluid-phase conductors can experience deformations between 20% and 40% relative to an “at rest” condition, thus varying electrical parameters generated by the circuit 1400.

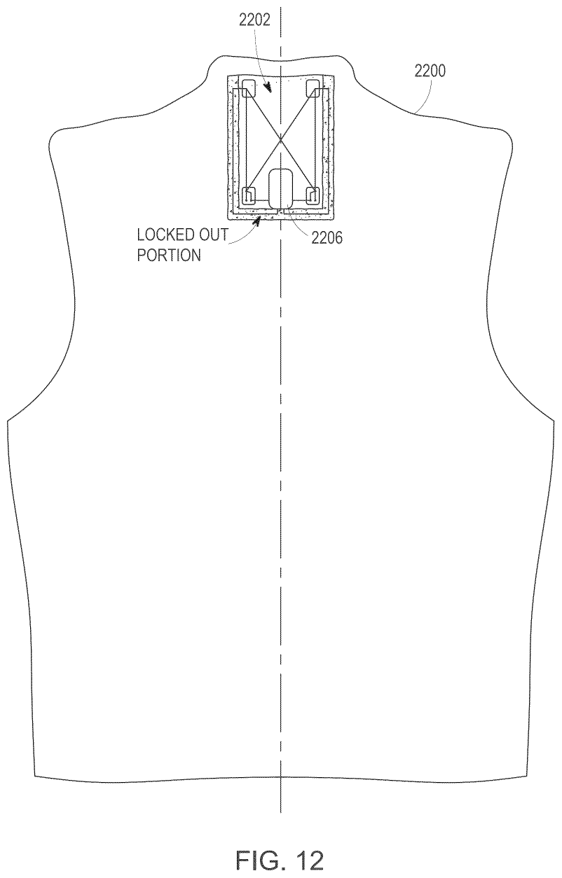

According to the non-limiting aspects where alternate conductors (e.g., silver ink, etc.) are used to form strain-sensing, flexible circuits, such circuits may experience no hysteresis and thus, may experience measurable changes in electrical characteristics upon returning to a relaxed state after undergoing a number of deformation cycles. This is known as “strain creep,” or a degradation in performance as the number of deformation cycles increases. According to such aspects, the performance of a strain sensing flexible circuit 1300 that utilizes such alternate conductors can be enhanced via various calibration methods, such as the method 1200 of FIG. 12, disclosed herein.

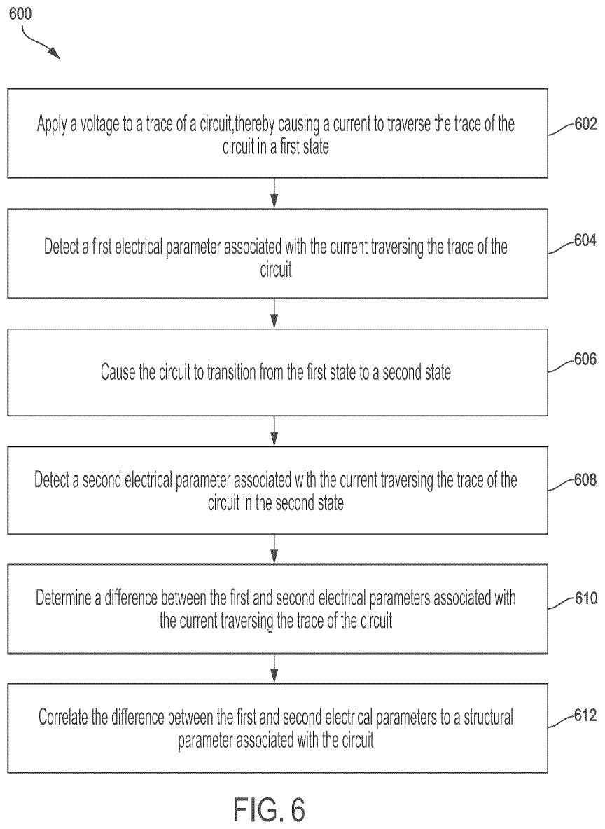

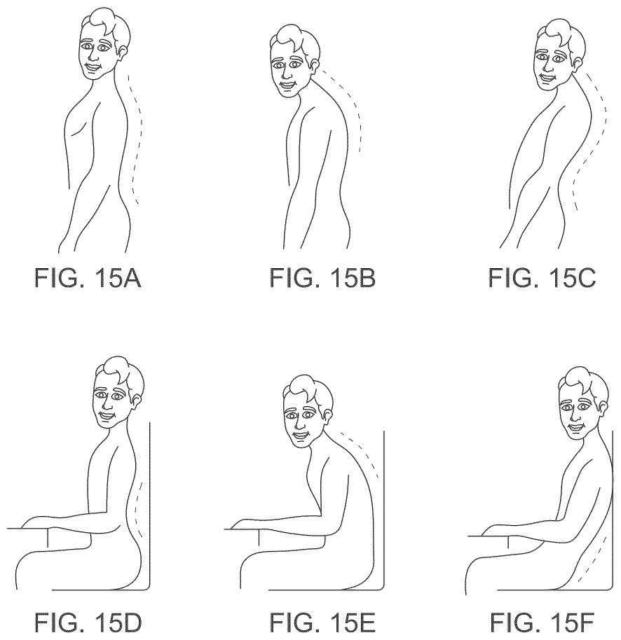

According to the non-limiting aspect of FIGS. 5A and 5B, the processor 1404 can receive signals from the various sensors and/or components dispositioned on the flexible circuit 1400 and thus, the processor 1404 can discern differences in generated electrical parameters and correlate them to various physical parameters associated with the deformation of the flexible circuit 1400, as disclosed in International Patent Application No. PCT/US2022/078810, titled DEVICES, SYSTEMS, AND METHODS FOR MAKING AND USING A FLUID-FILLABLE CIRCUIT, and filed Oct. 27, 2022, the disclosure of which is hereby incorporated by reference in its entirety. In summary, an electrical parameter (e.g., an inductance, a resistance, a voltage drop, a capacitance, and an electromagnetic field, etc.) of the circuits 1400, 1420, 1430 can be correlated to a structural parameter (e.g., a dimension, a strain, a stress, a pressure, etc.) of the circuits 1400, 1420, 1430. In other words, when the circuit 1400, 1420, 1430 is in an initial condition-such as an unstrained condition of FIG. 5A—the circuits 1400, 1420, 1430 may generate a first electrical parameter, but when placed in a second condition-such as a strained condition of FIG. 15B—the circuits may generate a second electrical parameter. Correlation of electrical parameters generated by the flexible circuits 1400, 1420, 1430 and a structural parameter of the flexible circuits 1400, 1420, 1430 will be described in further detail with reference to the method 600 of FIG. 6.

Referring now to FIGS. 5E and 5F, an electrode 1440 configured for use with the flexible circuits and wearable articles disclosed herein is depicted in accordance with at least one non-limiting aspect of the present disclosure. According to the non-limiting aspect of FIGS. 5E and 5F, the electrode 1440 can be formed from a flexible sheet such that the electrode 1440 has a domed curvature that produces a spring-like effect which can bias the electrode 1440 against the user's skin providing supplemental pressure and improved performance of the electrode 1440. Although shown here with a generally circular shape, it should be appreciated that any shape of electrode 1440 can be provided, assuming the shape facilitates the formation of a skin-contacting surface that can be biased against the skin of a user. Here, the major dimension may be the diameter D of the electrode. However, according to non-limiting aspects wherein the perimeter shape of the electrode 1440 is a square or rectangle, the Length or Width may have been determined to be the major dimension.

According to other non-limiting aspects, the electrode 1440 of FIGS. 5E and 5F can include a variety of other geometric configurations, including a flat, sheet of any shape (e.g., circle, rectangular, triangle, etc.), a “pellet” type configuration-similar to the domed electrode 1440 of FIGS. 5E and 5F but solid instead of hollow- or a “leaf spring” configuration molded or otherwise formed to have a radius of curvature that extends a single dimension of the electrode such that the electrode can be biased against the skin. For example, according to some non-limiting aspects, the electrode can be configured similar to any of those disclosed in International Patent Application No. PCT/US2023/062668, titled DEVICES, SYSTEMS, AND METHODS FOR CHARACTERIZING MOTIONS OF A USE VIA WEARABLE ARTICLES WITH FLEXIBLE CIRCUITS, and filed Feb. 15, 2023, the disclosure of which is herein incorporated by reference in its entirety.

Referring now to FIG. 6, a flow chart illustrating a method 600 of monitoring posture using a flexible circuit is depicted in accordance with at least one non-limiting aspect of the present disclosure. According to the non-limiting aspect of FIG. 6, the method 600 can include applying 602 a voltage to a trace of a flexible circuit in a first state, thereby causing a current to traverse the trace of the flexible circuit. Once a current is flowing through the circuit, the method 600 can include detecting 604 a first electrical parameter (e.g., an inductance, a resistance, a voltage drop, a capacitance, and an electromagnetic field, etc.) associated with the current traversing the trace of the flexible circuit in the first state. The method 600 can further include causing 606 the flexible circuit to transition from the first state to a second state. For example, causing 606 the transition can include the user bending or rotating. The method 600 can further include detecting 608 a second electrical parameter associated with the current traversing the trace of the flexible circuit and determining 610 a difference between the first and second electrical parameters associated with the current traversing the trace of the flexible circuit. Finally, the method can include correlating 612 the difference between the first and second electrical parameters to a structural parameter (e.g., a strain, a stress, a pressure, a dimension, etc.) associated with the flexible circuit. It shall be appreciated that, by using the varying electrical parameters to characterize the structural parameters of a flexible circuit, the physical characteristics of the wearable article and thus, a posture of the user can be characterized. Since different users will have varying heights, weights, and body shapes, it shall be appreciated that some degree of data normalization will be required to properly monitor and characterize each user's posture. However, the steps illustrated in FIG. 6 are not the exclusive steps of the method 600 contemplated by the present disclosure. For example, in an embodiment where the flexible circuit incorporates an electrode in contact with a mammalian body, rather than applying a voltage and/or current to the circuit, a similar method may be used to monitor a voltage or current that is applied to the circuit by neuromuscular activity through the electrode's contact with the body.

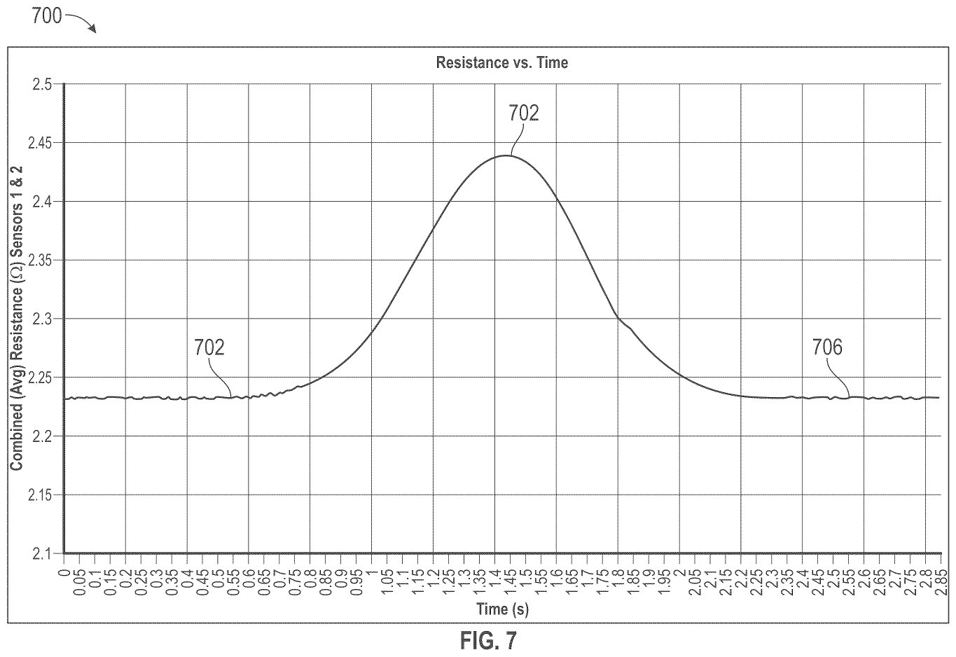

Referring now to FIG. 7, a chart 700 of an electrical parameter generated by a flexible circuit on an article worn by a user as their posture changes in time is depicted in accordance with at least one non-limiting aspect of the present disclosure. The flexible circuits used to generate the electrical parameter of FIG. 7 can include a strain sensor system 100 with a strain sensor 102, as described in reference to FIGS. 1-4. Likewise, the flexible circuits used to generate the electrical parameter of FIG. 7 can be configured similar to the flexible circuits 1400, 1420 depicted in FIGS. 5A-C. According to the non-limiting aspect of FIG. 7, a flexible circuit can be used to generate a varying electrical parameter, such as a resistance measured across the flexible circuit. However, according to other non-limiting aspects the electrical parameter can include an inductance, a voltage drop, a capacitance, and an electromagnetic field, amongst other electrical parameters that will vary based on the changing geometry of the flexible circuit.

As described in reference to the method 600 of FIG. 6, as a user may alter their posture by bending or rotating, thereby causing the flexible circuit to transition from a first state to a second state. As a result of the transition, the trace—and more specifically, the deformable conductor—may elongate and/or reduce in cross-sectional area, thereby altering one or more electrical parameters generated by the flexible circuit. For example, according to the non-limiting aspect of FIG. 7, at a first point in time 702 of about 0.6 seconds, the user's posture may be in a neutral position causing one or more flexible circuits to generate an average resistance of approximately 2.24 ohms. However, at a second point in time 704 of about 1.45 seconds, the user may have bent, or rotated. As a result of this motion, the deformable conductor may have elongated or reduced in cross-sectional area relative to the first point in time 702, thereby increasing the average resistance generated by one or more flexible circuits to approximately 2.44 ohms. At a third point in time 706 of about 2.35 seconds, the user may have returned to a neutral posture position, thereby causing the one or more flexible circuits to once again generate an average resistance of approximately 2.24 ohms. As such, the difference between a first and second electrical parameter generated by the flexible circuit in various stats can be correlated to a structural parameter (e.g., a strain, a stress, a pressure, a dimension, etc.) of the flexible circuit and used to monitor and/or characterize the motions or posture of the user.

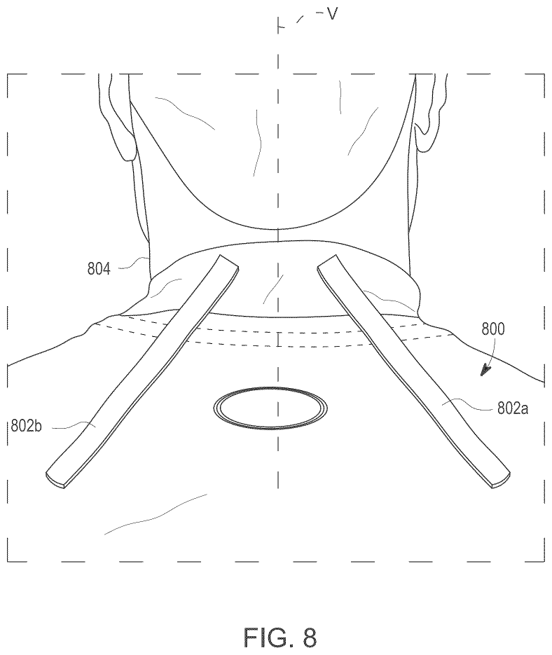

Referring now to FIG. 8, a wearable article 800 featuring flexible circuits 802ₐ, 802_{b} configured to monitor the posture of a user 804 is depicted in accordance with at least one non-limiting aspect of the present disclosure. The flexible circuits 802ₐ, 802_{b} can include a strain sensor system 100 with a strain sensor 102, as described in reference to FIGS. 1-4. Likewise, the flexible circuits 802ₐ, 802_{b} of the wearable article 800 of FIG. 8 can be configured similar to and can include any of the particular features or components as described in reference to the flexible circuit 1400, 1420, 1430 of FIGS. 5A-C. According to the non-limiting aspect of FIG. 8, the flexible circuits 802ₐ, 802_{b} are positioned relatively high on the wearer's 804 back, approximately just below the C7 vertebrae. Specifically, the flexible circuits 802ₐ, 802_{b} are configured for biaxial strain sensing. For example, the particular configuration of FIG. 8 can enable the monitoring of the trapezius, to track the user's 804 shrugging and/or other neck movements, as desired. However, according to other non-limiting aspects, the flexible circuits 802ₐ, 802_{b} can be positioned elsewhere on the wearable article 800 depending on user preference or intended application. For example, it may be beneficial to arrange the flexible circuits 802ₐ, 802_{b} in proximity to either the T3, T4, T5, T6, T8, T12, L1, L3, L5, S1, and S2 vertebrae depending on the particular point of the spine the user 804 wants to monitor.

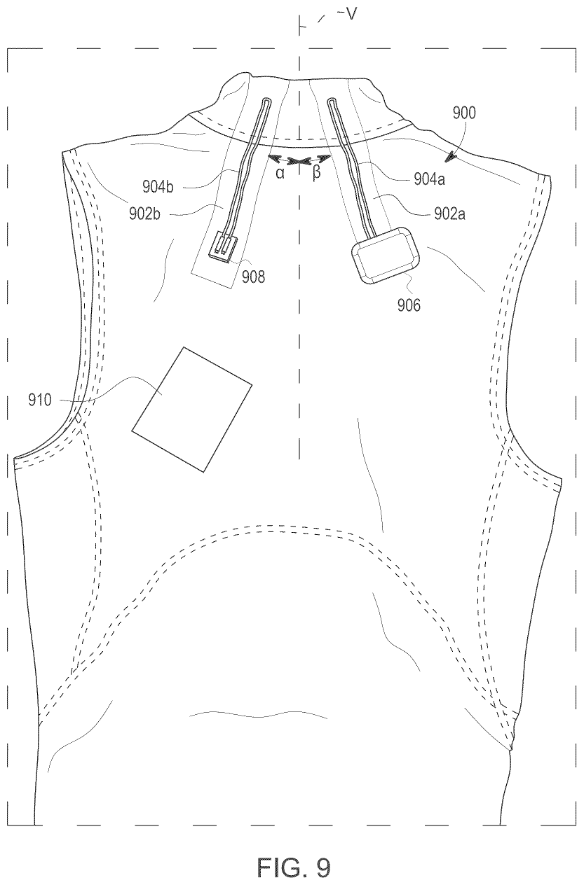

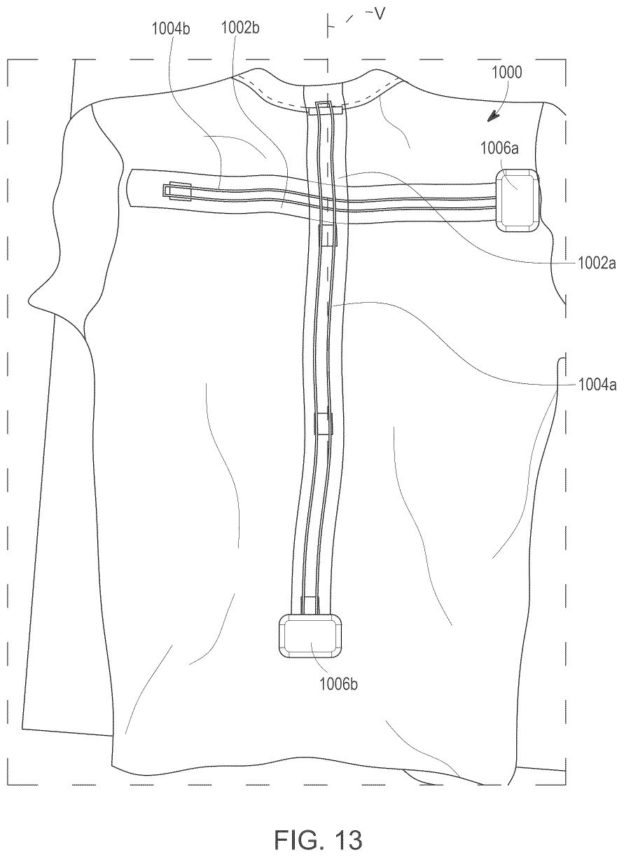

Additionally, it shall be appreciated that, according to the non-limiting aspect of FIG. 8, the arrangement of the flexible circuits 802ₐ, 802_{b} on the wearable article 800 can be attenuated for a desired monitoring and characterization. As previously discussed, the wearable article 800 can include two or more flexible circuits 802ₐ, 802_{b}. According to some non-limiting aspects, the first flexible circuit 802ₐ, can be oriented at a first angle that is greater than or equal to fifteen degrees and less than or equal to twenty five degrees relative to a vertical axis V defined by a neutral position of the user's 804 spine and the second flexible circuit 802_{b}, can be oriented at a second angle that is greater than or equal to fifteen degrees and less than or equal to twenty five degrees relative to the vertical axis V defining the opposite direction. For example, according to the non-limiting aspect of FIG. 8, a first flexible circuit 802ₐ can be oriented at twenty degrees relative to a vertical axis V defined by a neutral position of the user's 804 spine and the second flexible circuit 802_{b} can be oriented at negative twenty degrees relative to the vertical axis V. The particular configuration of FIG. 8 can be implemented to assess the symmetry and/or asymmetry of the spinal column and thus, the user's 804 posture. However, it shall be appreciated that, according to other non-limiting aspects, any number of flexible circuits can be alternately arranged. For example, according to the non-limiting aspect of FIG. 13, each of the first and second flexible circuits 1002ₐ 1002_{b} can be perpendicularly arranged on a wearable article 1000, such that the first flexible circuit 1002ₐ is oriented at zero degrees relative to the vertical axis V and the second flexible circuit 1002_{b} is oriented at ninety degrees relative to the vertical axis V. as shown. As will be described in further detail with reference to the non-limiting aspect of FIG. 9, data associated with signals generated by the flexible circuits 802ₐ, 802_{b} on the wearable article 800 can be actively monitored, stored, processed independently, and/or compared to determine a relative position of the flexible circuits 802ₐ, 802_{b} in time, to generate a characterization of the user's posture.

In further reference to the non-limiting aspect of FIG. 9, the details and construction of a wearable article 900 featuring flexible circuits 902ₐ, 902_{b} configured to monitor the posture of a user are depicted in further detail. Ideally, the flexible circuits 902ₐ, 902_{b} would be adhered directly to the skin. However, it shall be appreciated that such a configuration could be impractical and/or uncomfortable for a user, especially if posture is to be monitored continuously for extended periods of time. Thus, the flexible circuits 902ₐ, 902_{b} can be adhered, laminated, or woven into the wearable article 900 of FIG. 9, which can be compressive such that the flexible circuits 902ₐ, 902_{b} are pressed firmly against the user's skin. The wearable article 900 can be constructed from a durable, lightweight, compressive material, such as a polyester, lycra, spandex and/or any other material suitable for a compressive, base layer. According to the non-limiting aspect of FIG. 9, the wearable article 900 can be a mock-neck crew or turtle neck, which could be beneficial for alleviating chaffing that may result when users frequently move their arms. Particularly, a mock or turtle neck can enable the flexible circuits 902ₐ, 902_{b} to be properly positioned at the top of a user's spinal column or at the bottom of their neck. However, it shall be appreciated that, according to other non-limiting aspects, the wearable article 900 can be manufactured from any other material and configured as any other type of shirt depending on user preference and/or intended application. In fact, according to other non-limiting aspects, the wearable article 900 can be configured as other types of clothing. For example, it might be beneficial to utilize a pant-like article with flexible circuits alone or in conjunction with the wearable article 900 of FIG. 9 to further characterize the user's posture.

Still referring to FIG. 9, traces 904ₐ, 904_{b} of the flexible circuits 902ₐ, 902_{b} can be formed from deformable conductors configured similar to the deformable conductors 1402 of FIGS. 5A-D. The traces 904ₐ, 904_{b} can terminate at and electrically couple to one or more electronic components 908 (e.g., processor, memory, transceiver, power source, light emitting diodes (“LEDs”), transducers, haptic sensors, connectors, contacts, etc.). The one or more electronic components 908 can be individually coupled to the flexible circuits 902ₐ, 902_{b}, incorporated into a single integrated circuit coupled to the flexible circuits 902ₐ, 902_{b}, or packaged within a modular and selectively removable housing 906. The one or more electronic components 908 can be configured to receive, store, process, and/or otherwise react to signals received from the flexible circuits 902ₐ, 902_{b}. According to some non-limiting aspects, the one or more electronic components 908 are packaged within a housing they can be selectively coupled to the flexible circuits 902ₐ, 902_{b}. of the wearable article 900 via a mechanical cradle mounted on the wearable article. For example, according to some non-limiting aspects, the modular, selectively removable electronic component 906 configured similar to the power components described in U.S. Provisional Patent Application No. 63/412,867, titled DEVICES, SYSTEMS, AND METHODS TO MONITOR AND CHARACTERIZE THE MOTIONS OF A USER VIA FLEXIBLE CIRCUITS, and filed on Oct. 3, 2022, the disclosure of which is hereby incorporated by reference in its entirety.

According to some non-limiting aspects, the one or more electronic components 908 of the wearable article 900 of FIG. 9 can be configured for onboard signal processing and/or transmission. For example, according to some non-limiting aspects, the one or more electronic components 908 can include a microprocessor (e.g., a Nordic-brand nRF MDK-based processor or equivalent, etc.), a memory, a wireless communication circuit, and/or a bus port (configured to receive power and/or data from a power component of the electronic component 908), an additional IMU, and/or additional sensors, amongst other electronic components. According to some non-limiting aspects, an analog-to-digital converter (“ADC”), for example, can be positioned on one or more electronic components 908.

According to other non-limiting aspects, the one or more electronic components 908 can include a power source, such as a battery and/or a charger. The charger, for example, can include a universal serial bus (“USB”) port configured to convey electrical power and/or data to the one or more electronic components 908 from an external source. For example, the one or more electronic components 908 can be configured for such conveyance via a USB-A, USB-B, or USB-C protocol, although other means for power and/or data conveyance are contemplated by the present disclosure. According to other non-limiting aspects, the one or more electronic components 908 can include a wireless charging circuit and/or a wireless transmitter and/or receiver configured to wireless obtain power and data from external sources. Regardless, it shall be appreciated that the one or more electronic components 908, when mechanically and electrically coupled to the wearable article 900, can provide electrical power to the one or more electronic components 908 and/or flexible circuits 902ₐ, 902_{b}. Additionally, via the one or more electronic components 908, it shall be appreciated that data can be transmitted to and from the flexible circuits 902ₐ, 902_{b}. For example, according to some non-limiting aspects, the one or more electronic components 908 can be used to transmit a firmware update to a memory of the wearable article 900, for execution by a microprocessor. Alternately, the one or more electronic components 908 can include a memory configured to store data generated by the flexible circuits 902ₐ, 902_{b} for subsequent use and processing.

As previously discussed, the wearable article 900 can include a mechanical component, such as a cradle, configured to removably secure the housing 906 containing one or more electronic components to the wearable article 900. Accordingly, the cradle can establish electrical communication between the one or more electronic components 908 and the flexible circuits 902ₐ, 902_{b}, of the wearable article 900 of FIG. 9. According to still other non-limiting aspects, the electronic component 2808 can include a memory and/or transceiver. Thus, when the electronic component 2808 is mechanically secured to the wearable article 2800 via the cradle, the electronic component 2808 can provide power and/or data to the other electronics of the glove 2800. Specifically, the electronic component 908 or housing 906 can include a microprocessor configured to process signals received from the flexible circuits 902ₐ, 902_{b} and perform one or more steps of the method 600 of FIG. 6. However, according to other non-limiting aspects the electronic component 908 can include a transmitter configured to transmit signals received from the flexible circuits 902ₐ, 902_{b} to a remote processor and/or a display. According to still other non-limiting aspects, the electronic component 908 can include a memory configured to store signals received from the flexible circuits 902ₐ, 902_{b}.

Accord to other non-limiting aspects, the electronic component 908 of the wearable article 900 of FIG. 9 can include a feedback component, such as an LED, a transducer, and/or a haptic feedback. If a processor determines that the user's posture has deviated from a baseline based on the signals received from the flexible circuits 902ₐ, 902_{b}, the feedback component can provide the user with feedback prompting the user to correct their posture. For example, one or more LEDs may be illuminated, wherein the quantity of LEDs are indicative of a degree of deviation from the baseline. Likewise, a haptic sensor can vibrate, providing the user with tactile feedback. Alternately, a transducer can emit an audible sound notifying the user that their posture has deviated from the baseline. Accordingly, the present disclosure contemplates numerous means providing the user with information regarding their current posture relative to a baseline posture. This can further include visual feedback provided by a user interface presented by either an onboard or remote display. According to the non-limiting aspect of FIG. 9, the wearable article 900 can further include a pocket 910 or alternate means of storing an auxiliary device that may be in physical, electrical or wireless communication with the wearable 900, e.g.: a battery or batteries; a smartphone or other computing device; a transmitter. In some embodiments, a cable or wires may connect the auxiliary device to at least one of the electrical components 908. As previously discussed, different users will have varying heights, weights, ages, capabilities, and body shapes. Therefore, it shall be appreciated that some degree of data normalization will be required to properly monitor and characterize each user's posture, especially when evaluating the user's posture against a predetermined baseline. For example, it shall be appreciated that evaluating the magnitude of a calculated delta against a predetermined baseline can provide an accurate assessment of the user's posture in consideration of their differing physical traits.

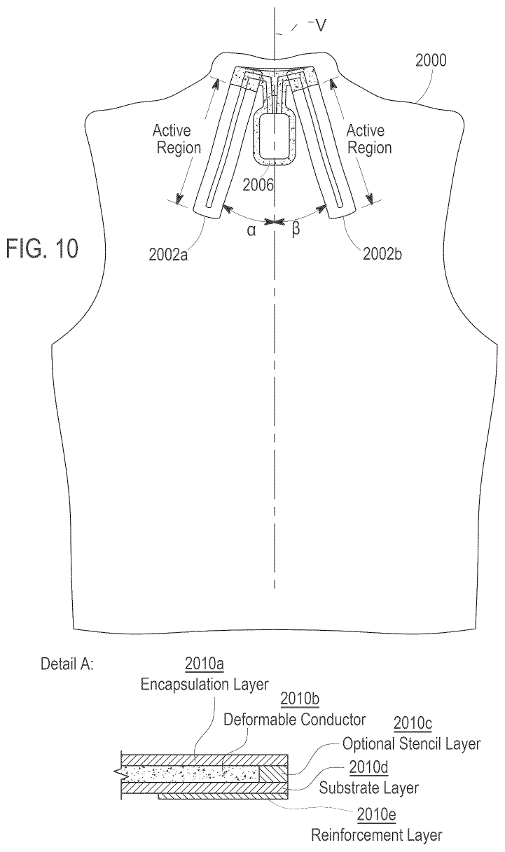

Referring now to FIGS. 10-12, several wearable articles 2000, 2100, 2200 featuring flexible circuits configured to monitor the posture of a user are depicted in accordance with several non-limiting aspects of the present disclosure. For example, according to the non-limiting aspect of FIG. 10, the wearable article 2000 can be configured as a fully contained system, comprising a first flexible circuit 2002ₐ and a second flexible circuit 2002_{b}, which are respectively oriented at a first angle α and a second angle β from the vertical axis V defined by the wearable article 2000. Although the first angle α can be equal to the second angle β, according to other non-limiting aspects, the first angle α can be different from the second angle β. Each of the first angle α and the second angle β can be specifically configured to ensure each of the flexible circuits 2002ₐ, 2002_{b} monitor a different facet of the user's posture.

The wearable article 2000 of FIG. 10 can include one or more electronic components (e.g., a processor, a memory, a transceiver, an inertial measurement units, a display, a power source, LEDs, a radio transceiver, and analog to digital converter, transducers, haptic sensors, connectors, contacts, etc.) coupled to the flexible circuits 2002ₐ, 2002_{b} and packaged within a housing 2006. According to some non-limiting aspects, the housing 2006 can be modular and selectively removable from a mechanical cradle mounted to the wearable article 2000.

Furthermore, as depicted in FIG. 10, a portion of the flexible circuits 2002ₐ, 2002_{b} can be configured to function as an “active region” of the flexible circuits 2002ₐ, 2002_{b}, as these portions are positioned on a portion of the wearable article 2000 that is being targeted for monitoring. Conversely, the shaded portion of the flexible circuits 2002ₐ, 2002_{b}, including the portion on which the housing 2006 is mounted, can be “locked out.” As such, the locked out portions can include a substrate layer that is reinforced, for example, by one or more additional layers of the wearable article 2000, such that motion in the locked out portions is restricted, reduced, and/or eliminated, as desired. For example, one such configuration of a locked out portion of the flexible circuits 2002ₐ, 2002_{b} is depicted in Detail A. As previously discussed, a layup forming the flexible circuits 2002ₐ, 2002_{b} can include one or more layers surrounding a deformable conductor 2010_{b}, including an encapsulation layer 2010ₐ, an optional stencil layer 2010_{c}, and/or a substrate layer 2010ₑ. However, the locked out portions of the flexible circuits 2002ₐ, 2002_{b} can include a reinforcement layer 2010ₑ that is configured to stretch less relative to the rest of the layers 2010ₐ, 2010_{c}, 2010_{d}. For example, the reinforcement layer 2010ₑ can include any compatible, substantially non-stretchable material, such as marquisette fabric made from, e.g., cotton, nylon, polyester, rayon, or blends thereof. Accordingly, it shall be appreciated that this configuration can ensure that only a desired portion of the wearable article 2000 and thus, the user's posture is being monitored.

In some examples any or all portions of the flexible circuits 2002ₐ, 2002_{b} which are not intended to directly measure a posture indicative parameter may be locked out from stretching, as described above. For example, it may be advantageous to lock out portions containing traces intended as signal or power busses. Lock-out structures permit the circuits 2002ₐ, 2002_{b} to remain flexible, i.e., bendable and/or drape-able, but effectively limit stretching in the locked-out regions. This may provide additional benefits, such as amplifying the physical deformation of nonlocked-out regions in response to movements or poses assumed by the user of the wearable article 2000. When nonlocked-out regions are instrumented with a sensor, e.g., strain sensing traces 220X, 220Y, this may in turn amplify posture indicative parameter signal(s), e.g., resistance changes, produced by the sensors, which may be beneficial for, or more readily enable, detecting and/or computing and/or monitoring the user's posture via electronic components coupled to the flexible circuits 2002ₐ, 2002_{b}.

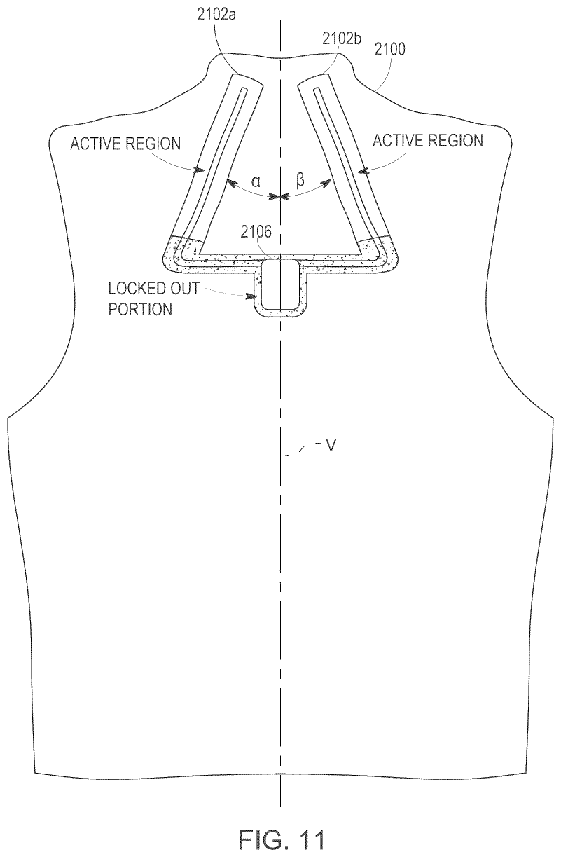

Referring now to FIG. 11, another wearable article 2100 featuring flexible circuits configured to monitor the posture of a user are depicted in accordance with a non-limiting aspect of the present disclosure. According to the non-limiting aspect of FIG. 11, the wearable article 2100 can include a first flexible circuit 2102ₐ and a second flexible circuit 2102_{b}, respectively oriented at a first angle α and a second angle β from the vertical axis V defined by the wearable article 2100. Once again, each of the first angle α and the second angle β can be specifically configured to ensure each of the flexible circuits 2102ₐ, 2102_{b} monitor a different facet of the user's posture. The wearable article 2100 can include one or more electronic components (e.g., a processor, a memory, a transceiver, an inertial measurement units, a display, a power source, LEDs, a radio transceiver, and analog to digital converter, transducers, haptic sensors, connectors, contacts, etc.) coupled to the flexible circuits 2102ₐ, 2102_{b} and packaged within a housing 2106. Similar to the wearable article 2000 of FIG. 10, according to some non-limiting aspects, the housing 2106 can be modular and selectively removable from a mechanical cradle mounted to the wearable article 2100 of FIG. 11. Additionally, the shaded portions of the circuits 2102ₐ, 2102_{b} are once again “locked out,” or partially restricted and reinforced, as discussed in reference to Detail A of FIG. 10, while non-shaded portions of the circuits 2102ₐ, 2102_{b} are active regions.

Referring now to FIG. 12, another wearable article 2200 featuring a flexible circuit 2202 configured to monitor the posture of a user are depicted in accordance with a non-limiting aspect of the present disclosure. According to the non-limiting aspect of FIG. 12, the wearable article 2200 can include a flexible circuit 2202 configured similar to the two-dimensional strain sensor 102 of FIG. 1. The wearable article 2200 can include one or more electronic components (e.g., a processor, a memory, a transceiver, an inertial measurement units, a display, a power source, LEDs, a radio transceiver, and analog to digital converter, transducers, haptic sensors, connectors, contacts, etc.) coupled to the flexible circuit 2202 and packaged within a housing 2206. Similar to the wearable articles 2000, 2100 of FIGS. 10 and 11, according to some non-limiting aspects, the housing 2206 can be modular and selectively removable from a mechanical cradle mounted to the wearable article 2200 of FIG. 12. Additionally, the shaded portion of the circuit 2202 shows one example configuration of a “locked out” region for this flexible circuit configuration. The locked out portion may be at least partially restricted and reinforced, as discussed in reference to Detail A of FIG. 10. The non-shaded portion of the circuit 2202 can remain an active region.

Referring now to FIG. 13, another wearable article 1000 featuring flexible circuits 1002ₐ, 1002_{b} configured to monitor the posture of a user is depicted in accordance with at least one non-limiting aspect of the present disclosure. According to the non-limiting aspect of FIG. 10, a first flexible circuit 1002ₐ can be substantially aligned with—if not, parallel to, or oriented at a 0 degree angle to—the vertical axis V, which, in some examples may be configured to run generally along the spine of a wearer for the use case of a shirt for monitoring the posture of the wearer. A second flexible circuit 1002_{b} can be arranged substantially perpendicular to the first flexible circuit 1002ₐ, or at a 90 degree angle relative to the axis V, such that the first and second flexible circuits 1002ₐ, 1002_{b} are arranged in a cross-like configuration. Although the configuration of the flexible circuits 1002ₐ, 1002_{b} of FIG. 10 may not monitor the posture of the upper back and neck and may be less suitable for monitoring forward motions, the flexible circuits 1002ₐ, 1002_{b} of FIG. 10 may be implemented to monitor the posture of the lower back, abdomen, and/or arms. Alternatively, whereas each circuit 1002ₐ, 1002_{b} are shown as being configured as one large strain sensor, each circuit may comprise more than one strain or other sensor type along its length, and which couple to at least one electrical component. The electrical component may be any of the types mentioned above with reference to FIG. 9 and the circuits 902ₐ, 902_{b}. Further, the offset of the circuits 902ₐ, 902_{b}, 1002ₐ, 1002_{b} relative to the axes V may range anywhere from 0 to 180 degrees. Further still, while in each of the embodiments of FIGS. 9, 10 circuits 902ₐ, 902_{b}, 1002ₐ, 1002_{b} are shown as discrete elements, the various circuits may be adapted to be in physical, structural, and/or electrical communication with one another, and may share and each be in communication with a common electrical component. Accordingly, it shall be appreciated that the present disclosure contemplates numerous aspects in which wearable articles and flexible circuits are alternately configured to monitor a variety of different motions, body parts, and/or postures throughout a user's body.

As previously discussed, the flexible circuits 1002ₐ, 1002_{b} of the wearable article 1000 of FIG. 13 (or any of the wearable articles disclosed herein) can include traces 1004ₐ, 1004_{b} made from deformable conductors and communicably coupled to one or more electronic components (e.g., processor, transceiver, power source, memory, feedback components, etc.). According to the non-limiting aspect of FIG. 13, the flexible circuits 1002ₐ, 1002_{b} can be coupled to one or more electronic components (e.g., a processor, a memory, a transceiver, an inertial measurement units, a display, a power source, LEDs, a radio transceiver, and analog to digital converter, transducers, haptic sensors, connectors, contacts, etc.) packaged within one or more housings 1006ₐ, 1006_{b}. Similar to the wearable article 2000 of FIG. 10, according to some non-limiting aspects, one or more housings 1006ₐ, 1006_{b} can be modular and selectively removable from a mechanical cradle mounted to the wearable article 1000 of FIG. 13. For example, according to some non-limiting aspects, the flexible circuits 1002ₐ, 1002_{b} can transmit signals via one or more processors in the one or more housings 1006ₐ, 1006_{b} to a remote processor and/or display, which can process and present a computer-generated simulation of the user's posture based on signals generated by the flexible circuits 1002ₐ, 1002_{b}. For example, this can be done in accordance with the method 600 of FIG. 6. Moreover, it shall be appreciated that the fit of the wearable article 1000 on the user is important. However, different users will require the wearable article 1000 to be different sizes, depending on the weight, age, height, and/or body shape. Thus, the signals generated by the flexible circuits 1002ₐ, 1002_{b} can be processed in accordance with the method 600 of FIG. 6 and differences in magnitudes of generated electrical parameters can be utilized to accurately assess the user's posture against a predetermined baseline.

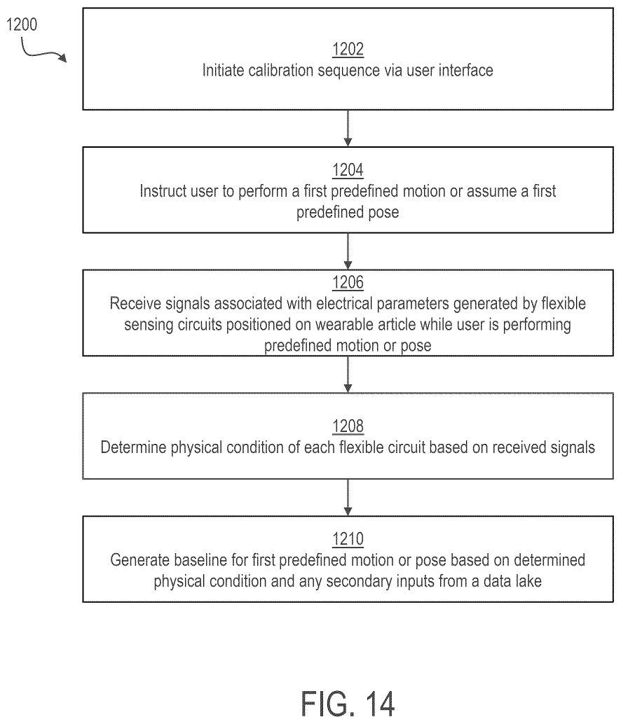

Referring now to FIG. 14, a method 1200 of calibrating data generated by the flexible circuits of a wearable article is depicted in accordance with at least one non-limiting aspect of the present disclosure. According to the non-limiting aspect of FIG. 14, the method 1200 can include initiating 1202 a calibration sequence via a user interface of an application executed or otherwise accessed by a processor or other computing device. The method 1200 can then include instructing 1204 a user wearing the wearable device to perform a first predefined motion or assume a first predefined pose and receiving 1206 signals associated with electrical parameters generated by flexible sensing circuits positioned on wearable article while user is performing predefined motion or pose. The method 1200 can further include determining 1208 a physical condition of each flexible circuit on the wearable article based on the received signals and generating 1210 a baseline for the first predefined motion or pose based on the determined physical condition. An instructed predefined pose may include the use of an object external to the user, e.g, standing erect against a wall or lying flat on a floor.

It should be further appreciated that the method 1200 of FIG. 14 can further include generating 1210 the baseline based on any secondary inputs, including those generated via artificial intelligence based on an aggregate pool of previously-generated data, such as a data lake. For example, the processor executing the calibration sequence of FIG. 14 can be communicably coupled to one or more servers configured to store such aggregated pools of data. Accordingly, an artificial intelligence algorithm can be implemented to utilize the signals generated by each flexible circuit on the wearable article to supplement and/or further inform conclusions drawn by the artificial intelligence algorithm. Likewise, the artificial intelligence algorithm can provide inputs to the generation of the baseline based on previously calculated averages for a particular user's age, body part, condition, etc. In other words, the baselines generated via the calibration method 1200 of FIG. 14 can be ever evolving and thus, become more accurate as the data lake becomes larger. According to some non-limiting aspects, the wearable articles 800, 900, 1000 disclosed herein can further include an inertial measurement unit (“IMU”) configured to measure three-dimensional rotational motions of the user. As such, the method 1200 of calibration depicted in FIG. 14 can further include a consideration of data generated by the IMU, as disclosed in U.S. Provisional Application No. 63/412,867, titled DEVICES, SYSTEMS, AND METHODS TO MONITOR AND CHARACTERIZE THE MOTIONS OF A USER VIA FLEXIBLE CIRCUITS, filed Oct. 3, 2022, the disclosure of which is hereby incorporated by reference in its entirety. For example, according to some non-limiting aspects, if a user is reaching with their hands but maintaining a neutral posture, the flexible circuits disclosed herein might register that motion as a change in the user's posture. As such, calibrating the wearable article with IMU inputs could account for and properly contextualize such motions so they do not falsely detect a change in the user's posture based on IMU data that indicates the user has a neutral posture.

Referring now to FIGS. 15A-F, several user interfaces displaying a generated baseline posture as well as a user's posture, as generated by a wearable article featuring flexible circuits, are depicted in accordance with at least one non-limiting aspect. For example, the user interface of FIG. 15A depicts a baseline posture. However, according to the user interface of FIG. 15B, the flexible circuits of the wearable article have detected that the user has a Kyphosis-type posture relative to the baseline of FIG. 15A, meaning the user has an exaggerated, forward rounding of the upper back. Likewise, according to the user interface of FIG. 15C, the flexible circuits of the wearable article have detected that the user has a Lordosis-type posture relative to the baseline of FIG. 15A, meaning the user has an exaggerated inward curve of the spine that typically affects the lower back. Similarly, the user interface of FIG. 15D depicts a baseline posture for the user while sitting and FIGS. 15E and 15F indicate that the flexible circuits of the wearable article have detected that the user is exhibiting a Kyphosis-type and Lordosis-type posture, respectively.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:

Clause 1: A system configured to monitor a posture of a user, the system including a wearable article including a first flexible circuit, wherein the first flexible circuit includes a first trace including a deformable conductor; and a computing device communicably coupled to the wearable article, wherein the computing device includes a processor and a memory configured to store instructions that, when executed by the processor, cause the processor to: receive a first signal from the first flexible circuit, wherein the first signal is corresponds to a physical deformation of the first trace; determine a first electrical parameter based on the first signal; determine the posture of the user based on the determined electrical parameter; compare the determined posture of the user to a baseline for the user's posture; and cause a display communicably coupled to the computing device to present a visual representation of the comparison.

Clause 2. The system according to clause 1, wherein, when executed by the processor, the instructions further cause the processor to transmit a signal configured to provide feedback to the user based on the comparison of the determined posture of the user to the baseline for the user's posture.

Clause 3. The system according to either of clauses 1 or 2, wherein the wearable article further includes a feedback component configured to provide the user with the feedback in response to the transmitted signal.

Clause 4. The system according to any of clauses 1-3, wherein the feedback component includes at least one of a light emitting diode, a haptic sensor, or a transducer, or combinations thereof.

Clause 5. The system according to any of clauses 1-4, wherein the baseline for the user's posture is calculated via an artificial intelligence algorithm based, at least in part, on a data lake including an aggregate of previously-generated data.

Clause 6. The system according to any of clauses 1-5, wherein, when executed by the processor, the instructions further cause the processor to update the baseline for the user's posture based on the received first signal.

Clause 7. The system according to any of clauses 1-6, wherein the wearable article further includes a second flexible circuit, wherein the second flexible circuit includes a second trace including a deformable conductor, and wherein, when executed by the processor, the instructions further cause the processor to receive a second signal from the second flexible circuit, wherein the second signal is corresponds to a physical deformation of the second trace, determine a second electrical parameter based on the second signal, and determine the posture of the user based on the determined second electrical parameter.

Clause 8. The system according to any of clauses 1-7, wherein the first flexible circuit is arranged at a twenty degree angle relative to a vertical axis defined by the wearable article, and wherein the second flexible circuit is arranged at a twenty degree angle relative to the vertical axis defined by the wearable article.

Clause 9. The system according to any of clauses 1-8, wherein the first flexible circuit is arranged at a twenty degree angle relative to a vertical axis defined by the wearable article, and wherein the second flexible circuit is arranged at a zero degree angle relative to the vertical axis defined by the wearable article.

Clause 10. The system according to any of clauses 1-9, wherein the first flexible circuit is arranged at a twenty degree angle relative to a vertical axis defined by the wearable article, and wherein the second flexible circuit is arranged perpendicular relative to the first flexible circuit.

Clause 11. A wearable article configured to monitor a posture of a user, the wearable article including a first flexible circuit, wherein the first flexible circuit includes a first trace including a deformable conductor, and wherein the wearable article is configured to be communicably coupled to a computing device, wherein the computing device includes a processor and a memory configured to store instructions that, when executed by the processor, cause the processor to receive a first signal from the first flexible circuit, wherein the first signal is corresponds to a physical deformation of the first trace, determine a first electrical parameter based on the first signal, determine the posture of the user based on the determined electrical parameter, compare the determined posture of the user to a baseline for the user's posture, and cause a display communicably coupled to the computing device to present a visual representation of the comparison.

Clause 12. The wearable article according to clause 11, further including a second flexible circuit, wherein the second flexible circuit includes a second trace including a deformable conductor, and wherein, when executed by the processor, the instructions further cause the processor to receive a second signal from the second flexible circuit, wherein the second signal is corresponds to a physical deformation of the second trace, determine a second electrical parameter based on the second signal, and determine the posture of the user based on the determined second electrical parameter.

Clause 13. The wearable article according to either of clauses 11 or 12, wherein the first flexible circuit is arranged at a twenty degree angle relative to a vertical axis defined by the wearable article, and wherein the second flexible circuit is arranged at a twenty degree angle relative to the vertical axis defined by the wearable article.

Clause 14. The wearable article according to any of clauses 11-13, wherein the first flexible circuit is arranged at a twenty degree angle relative to a vertical axis defined by the wearable article, and wherein the second flexible circuit is arranged at a zero degree angle relative to the vertical axis defined by the wearable article.

Clause 15. The wearable article according to any of clauses 11-14, wherein the first flexible circuit is arranged at a twenty degree angle relative to a vertical axis defined by the wearable article, and wherein the second flexible circuit is arranged perpendicular relative to the first flexible circuit.

Clause 16. A computer-implemented method of monitoring a posture of a user via a wearable article including a first flexible circuit, wherein the first flexible circuit includes a first trace including a deformable conductor, the method including receiving, via a processor, a first signal from the first flexible circuit, wherein the first signal is corresponds to a physical deformation of the first trace, determining, via the processor, a first electrical parameter based on the first signal, determining, via the processor, a posture of the user based on the determined electrical parameter, comparing, via the processor, the determined posture of the user to a baseline for the user's posture, and causing, via the processor, a display communicably coupled to the processor to present a visual representation of the comparison.

Clause 17. The computer-implemented method according to clause 16, further including transmitting, via the processor, a signal configured to provide feedback to the user based on the comparison of the determined posture of the user to the baseline for the user's posture.

Clause 18. The computer-implemented method according to either of clauses 16 or 17, further including calculating, via an artificial intelligence algorithm, the baseline for the user's posture based, at least in part, on a data lake including an aggregate of previously-generated data.

Clause 19. The computer-implemented method according to any of clauses 16-18, further including updating, via the processor, the baseline for the user's posture based on the received first signal.

Clause 20. The computer-implemented method according to any of clauses 16-19, wherein the wearable article further includes a second flexible circuit, wherein the second flexible circuit includes a second trace including a deformable conductor, and wherein the method further includes receiving, via the processor, a second signal from the second flexible circuit, wherein the second signal is corresponds to a physical deformation of the second trace determining, via the processor, a second electrical parameter based on the second signal, and determining, via the processor, the posture of the user based on the determined second electrical parameter.

Clause 21: The devices disclosed herein.

Clause 22: The systems disclosed herein.

Clause 23: The methods disclosed herein.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A system configured to monitor a posture of a user, the system comprising:
a wearable article comprising a first flexible circuit, wherein the first flexible circuit comprises a first trace comprising a deformable conductor; and
a computing device communicably coupled to the wearable article, wherein the computing device comprises a processor and a memory configured to store instructions that, when executed by the processor, cause the processor to:
receive a first signal from the first flexible circuit, wherein the first signal corresponds to a physical deformation of the first trace;
determine a first electrical parameter based on the first signal;
determine the posture of the user based on the determined electrical parameter; and
compare the determined posture of the user to a baseline for the user's posture.

2. The system of claim 1, wherein, when executed by the processor, the instructions further cause the processor to:
cause a display communicably coupled to the computing device to present a visual representation of the comparison.

3. The system of claim 2, wherein, when executed by the processor, the instructions further cause the processor to:
transmit a signal configured to provide feedback to the user based on the comparison of the determined posture of the user to the baseline for the user's posture.

4. The system of claim 3, wherein the wearable article further comprises a feedback component configured to provide the user with the feedback in response to the transmitted signal.

5. The system of claim 4, wherein the feedback component comprises at least one of a light emitting diode, a haptic sensor, or a transducer, or combinations thereof.

6. The system of claim 1, wherein the baseline for the user's posture is calculated via an artificial intelligence algorithm based, at least in part, on a data lake comprising an aggregate of previously-generated data.

7. The system of claim 6, wherein, when executed by the processor, the instructions further cause the processor to:
update the baseline for the user's posture based on the received first signal.

8. The system of claim 1, wherein the wearable article further comprises a second flexible circuit, wherein the second flexible circuit comprises a second trace comprising a deformable conductor, and wherein, when executed by the processor, the instructions further cause the processor to:
receive a second signal from the second flexible circuit, wherein the second signal corresponds to a physical deformation of the second trace;
determine a second electrical parameter based on the second signal; and
determine the posture of the user based on the determined second electrical parameter.

9. The system of claim 8, wherein the first flexible circuit is arranged at a twenty degree angle relative to a vertical axis defined by the wearable article, and wherein the second flexible circuit is arranged at a twenty degree angle relative to the vertical axis defined by the wearable article.

10. The system of claim 8, wherein the first flexible circuit is arranged at a twenty degree angle relative to a vertical axis defined by the wearable article, and wherein the second flexible circuit is arranged at a zero degree angle relative to the vertical axis defined by the wearable article.

11. The system of claim 8, wherein the first flexible circuit is arranged at a twenty degree angle relative to a vertical axis defined by the wearable article, and wherein the second flexible circuit is arranged perpendicular relative to the first flexible circuit.

12. A wearable article configured to monitor a posture of a user, the wearable article comprising:
a first flexible circuit, wherein the first flexible circuit comprises a first trace comprising a deformable conductor; and
wherein the wearable article is configured to be communicably coupled to a computing device, wherein the computing device comprises a processor and a memory configured to store instructions that, when executed by the processor, cause the processor to:
receive a first signal from the first flexible circuit, wherein the first signal is corresponds to a physical deformation of the first trace;
determine a first electrical parameter based on the first signal;
determine the posture of the user based on the determined electrical parameter;
compare the determined posture of the user to a baseline for the user's posture; and
cause a display communicably coupled to the computing device to present a visual representation of the comparison.

13. The wearable article of claim 12, further comprising a second flexible circuit, wherein the second flexible circuit comprises a second trace comprising a deformable conductor, and wherein, when executed by the processor, the instructions further cause the processor to:
receive a second signal from the second flexible circuit, wherein the second signal corresponds to a physical deformation of the second trace;
determine a second electrical parameter based on the second signal; and
determine the posture of the user based on the determined second electrical parameter.

14. The wearable article of claim 13, wherein the first flexible circuit is arranged at a first angle greater than or equal to fifteen degrees and less than or equal to twenty five degrees relative to a vertical axis defined by the wearable article, and wherein the second flexible circuit is arranged at a second angle greater than or equal to fifteen degrees and less than or equal to twenty five degrees relative to the vertical axis defined by the wearable article.

15. The wearable article of claim 13, wherein the first flexible circuit is arranged at about a twenty degree angle relative to a vertical axis defined by the wearable article, and wherein the second flexible circuit is arranged at about a twenty degree angle relative to the vertical axis defined by the wearable article.

16. The wearable article of claim 13, wherein the first flexible circuit is arranged at about a twenty degree angle relative to a vertical axis defined by the wearable article, and wherein the second flexible circuit is arranged at about a zero degree angle relative to the vertical axis defined by the wearable article.

17. The wearable article of claim 13, wherein the first flexible circuit is arranged at a twenty degree angle relative to a vertical axis defined by the wearable article, and wherein the second flexible circuit is arranged perpendicular relative to the first flexible circuit.

18. A computer-implemented method of monitoring a posture of a user via a wearable article comprising a first flexible circuit, wherein the first flexible circuit comprises a first trace comprising a deformable conductor, the method comprising:
receiving, via a processor, a first signal from the first flexible circuit, wherein the first signal corresponds to a physical deformation of the first trace;
determining, via the processor, a first electrical parameter based on the first signal;
determining, via the processor, a posture of the user based on the determined electrical parameter;
comparing, via the processor, the determined posture of the user to a baseline for the user's posture; and
causing, via the processor, a display communicably coupled to the processor to present a visual representation of the comparison.

19. The computer-implemented method of claim 18, further comprising:
transmitting, via the processor, a signal configured to provide feedback to the user based on the comparison of the determined posture of the user to the baseline for the user's posture.

20. The computer-implemented method of claim 18, further comprising:
calculating, via an artificial intelligence algorithm, the baseline for the user's posture based, at least in part, on a data lake comprising an aggregate of previously-generated data.

21. The computer-implemented method of claim 20, further comprising:
updating, via the processor, the baseline for the user's posture based on the received first signal.

22. The computer-implemented method of claim 18, wherein the wearable article further comprises a second flexible circuit, wherein the second flexible circuit comprises a second trace comprising a deformable conductor, and wherein the method further comprises:
receiving, via the processor, a second signal from the second flexible circuit, wherein the second signal is corresponds to a physical deformation of the second trace;
determining, via the processor, a second electrical parameter based on the second signal; and
determining, via the processor, the posture of the user based on the determined second electrical parameter.
